(19) United States
(12) Patent Application Publication
(10) Pub. No.: US 2020/0294622 A1
(43) Pub. Date: Sep. 17, 2020

(54) Subtyping of TNBC And Methods

(71) Applicant: NANTOMICS, LLC, Culver City, CA (US)

(72) Inventor: Christopher W. Szeto, Culver City, CA (US)

(21) Appl. No.: 16/765,462

(22) PCT Filed: Dec. 3, 2018
(86) PCT No.: PCT/US2018/063676

§ 371 Date: May 19, 2020

Related U.S. Application Data
(60) Provisional application No. 62/594,223, filed on Dec. 4, 2017.

(51) Int. Cl. G16B 25/10; G16B 20/00; G16B 40/30; G16B 40/20; G16H 10/40; G16H 70/60; G16H 20/10

(52) U.S. Cl. CPC G16B 25/10; G16B 20/00; G16B 40/30; G16H 20/10; G16H 10/40; G16H 70/60; G16B 40/20

(57) ABSTRACT

TBNC expression data are analyzed and subtyped into four distinct groups by expression level. Recursive feature elimination allowed for identification of about 80 genes that defined four clusters. So obtained cluster information can be used to associate the clusters with specific drug sensitivity, survival time, and other relevant parameters.

This application claims priority to our copending U.S. Provisional Patent Application with the Ser. No. 62/594,223, which was filed Dec. 4, 2017, which is incorporated by reference in its entirety herein.

### FIELD OF THE INVENTION

The field of the invention is characterizing breast cancer using omics analysis, especially as it relates to subtyping of breast cancer, especially TNBC (triple negative breast cancer).

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Treatment of patients with TNBC (breast cancer typically lacking expression of estrogen receptors, progesterone receptors and HER2 (human epidermal growth factor receptor 2)) is often challenging due to underlying genetic heterogeneity and the absence of well-defined molecular targets. TNBCs constitute 10%-20% of all breast cancers, and more frequently affect younger patients. TNBC tumors are typically larger in size, tend to have a higher grade and lymph node involvement, and are often more aggressive. Despite having higher rates of clinical response to presurgical (neoadjuvant) chemotherapy, TNBC patients have a higher rate of distant recurrence and a poorer prognosis than women with other breast cancer subtypes. Indeed, less than 30% of women with metastatic TNBC survive 5 years, and almost all patients die of breast cancer even with adjuvant chemotherapy.

More recently, efforts have been undertaken to refine TNBC into molecular subtypes into several molecularly distinct subgroups based on retrospective analysis of observed treatment responses to chemotherapy (see e.g., PLOS ONE|DOI:10.1371/journal.pone.0157368 Jun. 16, 2016). Similarly, subtypes for TNBC were defined based on five potential clinically actionable groupings of TNBC: 1) basal-like TNBC with DNA-repair deficiency or growth factor pathways; 2) mesenchymal-like TNBC with epithelial-to-mesenchymal transition and cancer stem cell features; 3) immune-associated TNBC; 4) luminal/apocrine TNBC with androgen-receptor overexpression; and 5) HER2-enriched TNBC (see e.g., Oncotarget, Vol. 6, No. 15; pp 12890-12908). In yet another study (see e.g., J Breast Cancer 2016 September; 19(3): 223-230), subtypes of TNBC were identified as basal-like, mesenchymal, luminal androgen receptor, and immune-enriched. In still further known studies, expression subtyping was performed and identified three sub-clusters among tested patient samples (see e.g., Breast Cancer Research (2015) 17:43). Likewise, an online classification tool was published to classify TNBC by gene expression (URL: cbc.mc.vanderbilt.edu/tnbc; Cancer Informatics 2012:11 147-156) that separated TNBC data into six distinct subtypes.

While such known methods provide at least some insight into different subgroups of TNBC, several of these subtypes are bound to specific parameters such as specific drug response, biomarkers, etc. and as such have an inherent bias. On the other hand, other methods require analysis of a substantially complete omics data set to identify a subtype. Consequently, analysis is often time consuming and expensive.

Despite remarkable advances in molecular insight into breast cancer genetics of TNBC, prediction of survival time or treatment success remains elusive. Therefore, there is still a need for improved systems and methods to better characterize TNBC subtypes that may help identify appropriate treatment methods and/or predict patient survival. Ideally, such improved systems and methods will not require a full omics data set but can be performed using a limited number of omics data.

### SUMMARY OF THE INVENTION

The inventive subject matter is directed to various systems and methods of omics analysis and especially expression analysis of a limited set of genes from a breast cancer sample that are suitable to identify TBNC and a particular molecular subtype within TBNC. Advantageously, such analysis is not tied to a particular outcome (e.g., treatment sensitivity or survival) and will require less than 100, and more typically less than 80 data for gene expression of selected genes.

Thus, in one aspect of the inventive subject matter, the inventor contemplates a method of processing omics data of a cancer sample that includes a step of obtaining transcriptomic data of a cancer tissue. Most preferably, the transcriptomics data is associated with protein expression level of a plurality of proteins in the cancer tissue, and the plurality of proteins is associated with a phenotype of the cancer tissue. Then, the transcriptomics data is stratified into a subgroup of data and the subgroup of data is clustered. In yet another step, the clustered subgroup of data is subjected to a recursive feature elimination to thereby obtain a reduced transcriptomic data.

For example, contemplated cancer samples include a breast cancer sample in which the plurality of proteins includes an estrogen receptor, a progesterone receptor, and HER2. In such example, the derived phenotype of the cancer tissue will be TNBC. However, other contemplated proteins include DNA repair proteins, cell cycle proteins, and/or proteins encoded by a cancer driver gene. Most typically, the transcriptomic data are RNAseq data, and/or the step of stratifying uses a cutoff value that is optimized for a ratio between true positive and false negative.

While not limiting to the inventive subject matter, the step of clustering may use between 3 and 10 clusters, and the recursive feature elimination is repeated at least once. Consequently, the reduced transcriptomic data are less than 30%, or less than 10%, or less than 1% of the transcriptomic data of a cancer tissue.

Where desired, contemplated methods may include a step of associating the reduced transcriptomic data with a drug response, overall survival, disease free survival, and/or progression free survival. In such embodiments, the method may further include a step of determining a treatment regimen based on at least one of the drug response, the overall survival, the disease free survival, and the progression free survival. Additionally, the method may also further include a step of treating a patient having the cancer tissue with a cancer treatment in the treatment regimen in a dose and a schedule sufficient to treat the cancer tissue. Moreover, the reduced transcriptomic data may also be used as an input for a pathway analysis.

In another aspect of the inventive subject matter, the inventors contemplate a system for processing omics data of a cancer tissue that includes an omics database storing transcriptomic data of the cancer tissue and a machine learning system informationally coupled to the omics database. The machine learning system is programmed to obtain the transcriptomic data of the cancer tissue, wherein the transcriptomics data is associated with protein expression level of a plurality of proteins in the cancer tissue, and wherein the plurality of proteins is associated with a phenotype of the cancer tissue, stratify the transcriptomics data into a subgroup of data, and clustering the subgroup of data, and subject the clustered subgroup of data to recursive feature elimination to obtain reduced transcriptomic data.

For example, contemplated cancer samples include a breast cancer sample in which the plurality of proteins includes an estrogen receptor, a progesterone receptor, and HER2. In such example, the derived phenotype of the cancer tissue will be TNBC. However, other contemplated proteins include DNA repair proteins, cell cycle proteins, and/or proteins encoded by a cancer driver gene. Most typically, the transcriptomic data are RNAseq data, and/or the step of stratifying uses a cutoff value that is optimized for a ratio between true positive and false negative.

While not limiting to the inventive subject matter, the subgroup is clustered using between 3 and 10 clusters, and the recursive feature elimination is repeated at least once. Consequently, the reduced transcriptomic data are less than 30%, or less than 10%, or less than 1% of the transcriptomic data of a cancer tissue.

Where desired, the machine learning system may be further programmed to associate the reduced transcriptomic data with a drug response, overall survival, disease free survival, and/or progression free survival. In such embodiments, the machine learning system may be further programmed to determine a treatment regimen based on at least one of the drug response, the overall survival, the disease free survival, and the progression free survival. Moreover, the reduced transcriptomic data may also be used as an input for a pathway analysis.

In still another aspect of the inventive subject matter, the inventors contemplate a non-transient computer readable medium that is informationally coupled to an omics database that stores transcriptomic data of a cancer tissue. The transient computer readable medium contains program instructions for causing a computer system comprising a machine learning system to perform a method of obtaining the transcriptomic data of the cancer tissue, wherein the transcriptomics data is associated with protein expression level of a plurality of proteins in the cancer tissue, and wherein the plurality of proteins is associated with a phenotype of the cancer tissue, stratifying the transcriptomics data into a subgroup of data, and clustering the subgroup of data, and subjecting the clustered subgroup of data to recursive feature elimination to obtain reduced transcriptomic data.

For example, contemplated cancer samples include a breast cancer sample in which the plurality of proteins includes an estrogen receptor, a progesterone receptor, and HER2. In such example, the derived phenotype of the cancer tissue will be TNBC. However, other contemplated proteins include DNA repair proteins, cell cycle proteins, and/or proteins encoded by a cancer driver gene. Most typically, the transcriptomic data are RNAseq data, and/or the step of stratifying uses a cutoff value that is optimized for a ratio between true positive and false negative.

While not limiting to the inventive subject matter, the step of clustering may use between 3 and 10 clusters, and the recursive feature elimination is repeated at least once. Consequently, the reduced transcriptomic data are less than 30%, or less than 10%, or less than 1% of the transcriptomic data of a cancer tissue.

Where desired, contemplated methods may include a step of associating the reduced transcriptomic data with a drug response, overall survival, disease free survival, and/or progression free survival. In such embodiments, the method may further include a step of determining a treatment regimen based on at least one of the drug response, the overall survival, the disease free survival, and the progression free survival. Moreover, the reduced transcriptomic data may also be used as an input for a pathway analysis.

Various objects, features, aspects and advantages of the inventive subject matter will become more apparent from the following detailed description of preferred embodiments, along with the accompanying drawings.

### BRIEF DESCRIPTION OF THE DRAWING

FIG. 1 is an exemplary mutation profile in most frequently mutated genes in breast cancer patients.

FIG. 2 is an exemplary graph depicting expression levels for various receptors on breast cancer cells vis-à-vis immunohistochemical status of receptor expression.

FIG. 3 provides exemplary graphs plotting true positive rate (TPR) versus false positive rate (FPR) as a function of cutoff values (in TPM) and associated accuracies at the selected cutoff values.

FIG. 4 depicts comparative results between immunohistochemical data (IHC) and RNAseq data for two selected receptors.

FIG. 5 depicts raw data for expression from two different study groups.

FIG. 6A is a graph plotting inconsistency versus number of subgroups.

FIG. 6B shows an exemplary heat map from 115 samples predicted as TNBC, and top 10K most variant genes.

FIG. 7 is an exemplary graph depicting best accuracies as a function of number of subgroups and gene set size.

FIG. 8 is an exemplary heat map of a minimal gene set for four TNBC subtypes.

### DETAILED DESCRIPTION

The inventors have now discovered that breast cancer can be accurately typed as triple negative breast cancer (TNBC) using expression data for selected receptor genes at appropriate threshold (i.e., cutoff) values and even subtyped into four distinct classes using expression data for a relatively small number of selected genes. Viewed from a different perspective, the inventors discovered that accurate diagnosing and/or characterizing the subtypes of breast cancers, especially TNBC can be performed with substantially reduced types and size of omics data when such reduced omics data is selected by clustering the data and eliminating less relevant data (e.g., via ranking the data based on the model and attributes, etc.). Thus, in one especially preferred aspect of the inventive subject matter, the inventors contemplate a method of processing omics data of a cancer tissue to obtain the reduced omics data set for subtyping the cancer tissue. In this method, transcriptomic data of the cancer tissue can be obtained and stratified into a subgroup of data, which is then clustered. Then, such clustered subgroup of data can be subjected to recursive feature elimination to obtain reduced transcriptomic data.

As used herein, the term “tumor” or “cancer” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use. As used herein, the term “bind” refers to, and can be interchangeably used with a term “recognize” and/or “detect”, an interaction between two molecules with a high affinity with a K_{D} of equal or less than 10⁻⁶M, or equal or less than 10⁻⁷M. As used herein, the term “provide” or “providing” refers to and includes any acts of manufacturing, generating, placing, enabling to use, or making ready to use.

As used herein, the term “locus” (or in plural, “loci”) refers to a portion of or a location in a gene, a transcript of a gene, or a nucleic acid molecule derived from a gene or a transcript of a gene.

It should be noted that any language directed to a computer should be read to include any suitable combination of computing devices, including servers, interfaces, systems, databases, agents, peers, engines, modules, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

Obtaining Omics Data: Any suitable methods and/or procedures to obtain omics data are contemplated. For example, the omics data can be obtained by obtaining tissues from an individual and processing the tissue to obtain DNA, RNA, protein, or any other biological substances from the tissue to further analyze relevant information. In another example, the omics data can be obtained directly from a database that stores omics information of an individual.

Where the omics data is obtained from the tissue of an individual, any suitable methods of obtaining a tumor sample (tumor cells or tumor tissue) or healthy tissue from the patient are contemplated. Most typically, a tumor sample or healthy tissue sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue. For example, tissues or cells may be fresh or frozen. In other example, the tissues or cells may be in a form of cell/tissue extracts. In some embodiments, the tissues or cells may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. In another example, a healthy tissue or matched normal tissue (e.g., patient's non-cancerous breast tissue) of the patient can be obtained from any part of the body or organs, preferably from liver, blood, or any other tissues near the tumor (in a close anatomical distance, etc.).

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of anti-tumor treatment (e.g., radiotherapy, chemotherapy, immunotherapy, etc.). In still another example, the tumor samples (or suspected tumor samples) may be obtained during the progress of the tumor upon identifying a new metastasized tissues or cells.

From the obtained tumor samples (cells or tissue) or healthy samples (cells or tissue), DNA (e.g., genomic DNA, extrachromosomal DNA, etc.), RNA (e.g., mRNA, miRNA, siRNA, shRNA, etc.), and/or proteins (e.g., membrane protein, cytosolic protein, nucleic protein, etc.) can be isolated and further analyzed to obtain omics data. Alternatively and/or additionally, a step of obtaining omics data may include receiving omics data from a database that stores omics information of one or more patients and/or healthy individuals. For example, omics data of the patient's tumor may be obtained from isolated DNA, RNA, and/or proteins from the patient's tumor tissue, and the obtained omics data may be stored in a database (e.g., cloud database, a server, etc.) with other omics data set of other patients having the same type of tumor or different types of tumor. Omics data obtained from the healthy individual or the matched normal tissue (or healthy tissue) of the patient can be also stored in the database such that the relevant data set can be retrieved from the database upon analysis. Likewise, where protein data are obtained, these data may also include protein activity, especially where the protein has enzymatic activity (e.g., polymerase, kinase, hydrolase, lyase, ligase, oxidoreductase, etc.). As used herein, omics data includes but is not limited to information related to genomics, proteomics, and transcriptomics, as well as specific gene expression or transcript analysis, and other characteristics and biological functions of a cell.

In an especially preferred embodiment, the omics data that is used to characterize the tumor, especially breast cancer, in this inventive subject matter is transcriptomics data. The transcriptomics data includes sequence information and expression level (including expression profiling, copy number, or splice variant analysis) of RNA(s) (preferably cellular mRNAs) that is obtained from the patient, from the cancer tissue (diseased tissue) and/or matched healthy tissue of the patient or a healthy individual. There are numerous methods of transcriptomic analysis known in the art, and all of the known methods are deemed suitable for use herein (e.g., RNAseq, RNA hybridization arrays, qPCR, etc.). The suitable transcriptomics data may typically include absolute or relative strength of transcription, for example, expressed as transcription levels of genes in the first location relative to transcription levels of genes in normal tissue of first patient. Alternatively, or additionally, transcriptomics data may also be expressed as relative abundance (e.g., transcripts per million (TPM)). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

Preferably, the transcriptomics data set includes allele-specific sequence information and copy number information. In such embodiment, the transcriptomics data set includes all read information of at least a portion of a gene, preferably at least 10×, at least 20×, or at least 30×. Allele-specific copy numbers, more specifically, majority and minority copy numbers are calculated using a dynamic windowing approach that expands and contracts the window's genomic width according to the coverage in the germline data, as described in detail in U.S. Pat. No. 9,824,181, which is incorporated by reference herein. As used herein, the majority allele is the allele that has majority copy numbers (>50% of total copy numbers (read support) or most copy numbers) and the minority allele is the allele that has minority copy numbers (<50% of total copy numbers (read support) or least copy numbers).

It should be appreciated that one or more desired nucleic acids or genes may be selected for a particular disease (e.g., cancer, etc.), disease stage, specific mutation, or even on the basis of personal mutational profiles or presence of expressed neoepitopes. Alternatively, where discovery or scanning for new mutations or changes in expression of a particular gene is desired, RNAseq is preferred to so cover at least part of a patient transcriptome. Moreover, it should be appreciated that analysis can be performed static or over a time course with repeated sampling to obtain a dynamic picture without the need for biopsy of the tumor or a metastasis. Thus, in some embodiments, the desired nucleic acids or genes may include genes encoding at least one of a DNA repair protein, a cell cycle protein, a neoepitope, an immune-response related genes, a protein encoded by a cancer driver gene, or any genes that are known to be specifically mutated or their expressions are up- or down-regulated in the tumor cells, or during tumorigenesis. In addition, the desired nucleic acids or genes may include genes encoding proteins that are associated with a phenotype of the cancer tissue. Thus, those genes may include any genes mutated or differentially expressed in different types of tumor or related or attributed to the shape or behavior (e.g., prone to be metastasized, solid tumor, cell shape, morphology of tumor tissue, etc.). For example, where the tumor is a breast cancer, the desired genes may be an estrogen receptor, a progesterone receptor, and/or HER2.

Consequently, the transcriptomics data may be associated with one or more protein expression level(s) of one or more protein(s) in the cancer tissue. Viewed from different perspective, the transcriptomics data may be used to infer one or more protein expression level(s) of one or more protein(s) in the cancer tissue. For example, RNAseq data on PD-L1 in a tumor tissue may show 10× increased TPM compared to the normal tissue, and such data can be associated with increased PD-L1 protein expression in the tumor tissue. Alternatively, at least it can be inferred that the PD-L1 protein expression in the tumor tissue is increased when the RNAseq data on PD-L1 in a tumor tissue may show 10× increased TPM compared to the normal tissue.

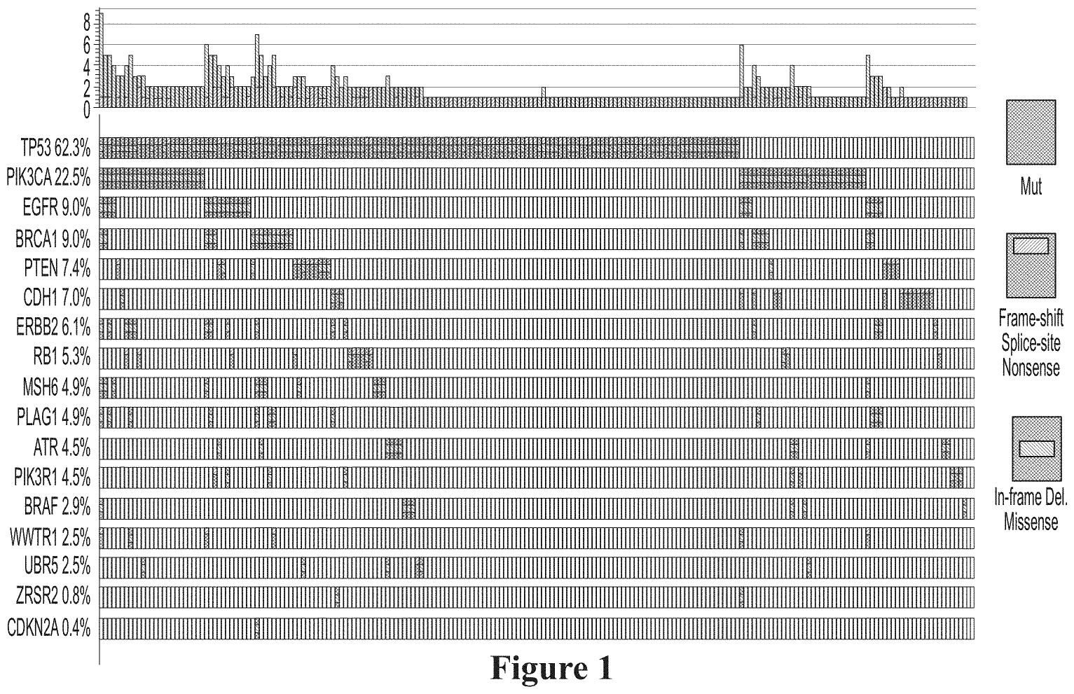

The inventors contemplate that types and/or scope of omics data that may be analyzed to classify the tumor or cancer may vary depending on the type of cancer or tumor of interest. For example, FIG. 1 illustrates most frequently mutated genes in the breast cancer tissues. Here, the top 20 most frequently mutated genes in breast cancer according to COSMIC (3 not shown due to zero-counts) are listed in rows, and each column represents one sample in one exemplary (here: GeparSepto) cohort. Grey boxes surround all non-WT genes, upper rectangular marks denote mutations that possibly disrupt the full-length transcript (e.g., nonsense mutations, frameshift mutation, mutations disrupting splicing), and lower rectangular marks denote in frame substitution mutations and/or missense mutations. As presence of various types of mutations varies among the cancer samples, mutational analysis to characterize cancer tissues for subtyping requires significant sequencing efforts and analytic time.

The inventors found that transcriptomics data of some genes, and/or inferred protein expression level from the transcriptomics data of some genes is more reliable to infer the status or classify a specific type of tumor. Viewed from different perspective, the inventors found that transcriptomics data of some genes, and/or inferred protein expression level from the transcriptomics data of some genes reflects the status or classify a specific type of tumor in more consistent and/or accurate manner. Thus, in an especially preferred embodiment, the inventors further contemplate that transcriptomics data of various genes can be stratified to identify the types of genes and their expression levels that can be more reliably used for characterizing the cancer tissue. While any suitable methods to stratify the transcriptomics data are contemplated, one preferred method uses a cutoff values that is optimized for a ratio between true positive and false negative values. Typically, the true positive and false negative values are determined based on the immunohistochemical data (IHC data) of the cancer tissues based on the known receptor status of the tumor tissue samples. In some embodiments, the transcriptomics data is stratified in a Youden plot in which the ratio of true positive to false positive was maximized. The so obtained cutoff values were cross validated in a 10-fold cross validation study using the same data and RNAseq data from an unrelated breast cancer cohort (e.g., TCGA, METABRIC, PRAEGNANT, etc.).

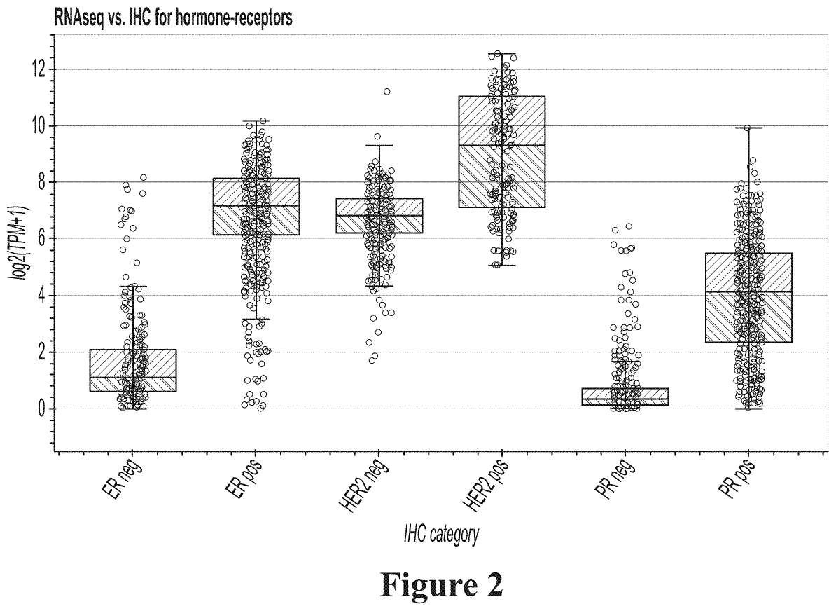

For example, TNBC status may be ascertained using RNAseq data (typically expressed as TPM (transcripts per million)) for the estrogen receptor, the progesterone receptor, and HER2. More particularly FIG. 2 exemplarily depicts a comparison of RNAseq data for the indicated receptors in a single patient cohort (TCGA BRCA).

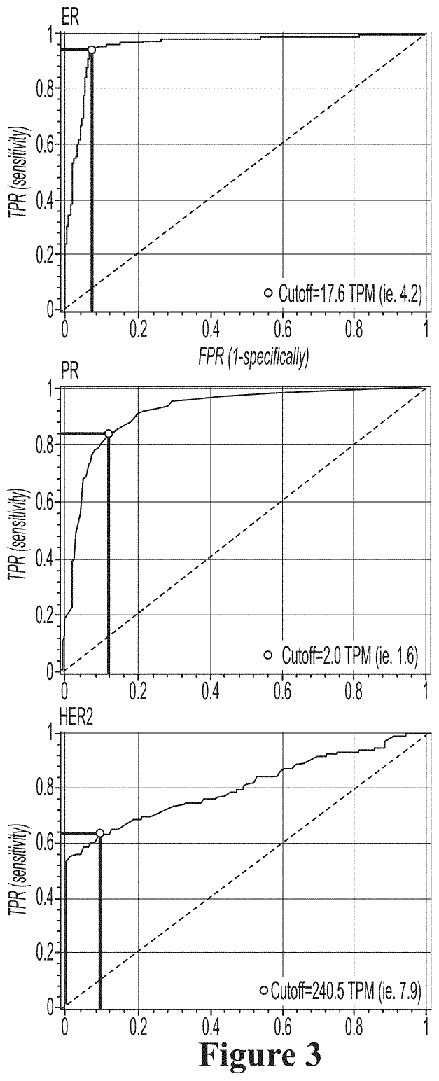

FIG. 3 show three Youden plots of receptor genes (ER, HR, and HER2) transcriptomics data plotted using true positive (TPR, sensitivity, y-axis) and false negative values (FPR, 1-specificity, x-axis). The threshold value was selected such that a ratio of true positive to false positive is maximized. Of course, it should be appreciated that cutoff values may also be derived from correlation with other manners of quantification, and especially with various mass spectroscopic methods (e.g., selected reaction monitoring type MS), which may achieve even tighter correlations.

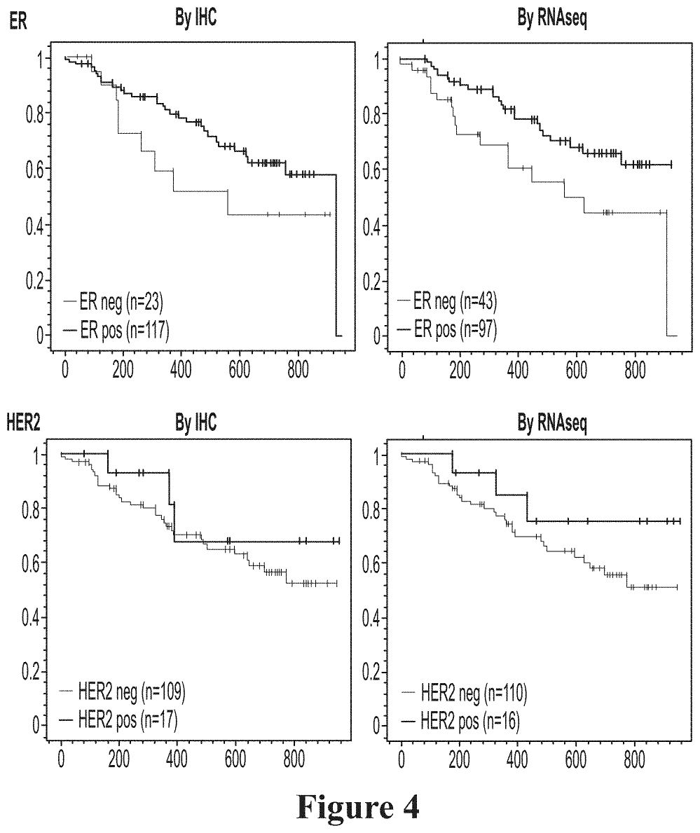

The so obtained cutoff values were cross validated in a 10-fold cross validation study using the same data and RNAseq data from an unrelated breast cancer cohort (PRAEGNANT). The inventors further found that the 10-fold cross-validation accuracy for all receptors (ER: 93.96%+/−1.28, PR: 84.18%+/−2.04, HER2: 84.56%+/−3.08), and accuracy in PRAEGNANT (ER: 83.33%, PR: 72.92%, HER2: 86.15%) are high across both cohorts. FIG. 4 exemplarily shows a parallel comparison between IHC results and RNAseq results for the ER and HER2 receptors using the so derived cutoff values in an independent cohort (PRAEGNANT) in order to validate and/or determine prognostic equivalence or superiority of RNAseq-based stratification.

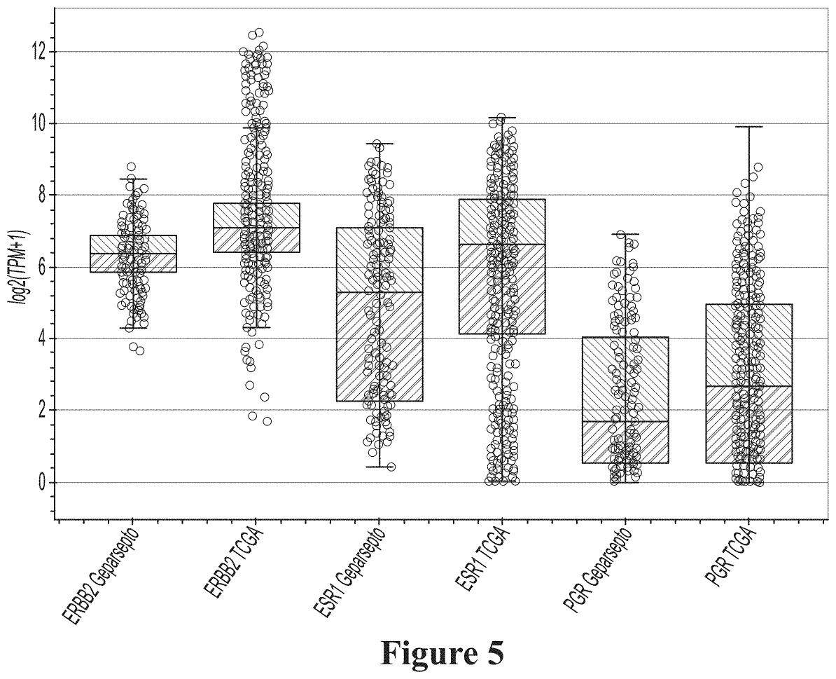

FIG. 5 shows another example of inferring protein expression levels of hormone receptors based on the RNAseq data and cross-validating such inferred data with the immunohistochemical data to determine the true positive/false negative ratio. Using the determined cutoff values for the respective receptors, a relatively large patient population from two distinct cohorts (GeparSepto and TCGA BRCA) was analyzed. Representative RNAseq data for the HER2, ER, and PR are shown in FIG. 5. This larger and well-defined dataset was then used to infer the likely status for each receptor, and Table 1 below shows the determination of receptor status using the so derived cutoff values on data of the GeparSepto cohort. The number of GeparSepto samples that are inferred as positive/negative for each hormone receptor (ER, PR, HER2) as well as the number inferred to be TNBC are provided. The inventors note that the proportion of TNBC samples (about 41%) is higher than the proportion within a randomized breast cancer population (10-20%), possibly due to the GeparSepto trial design of preselecting HER2- patients.

TABLE 1

| | ER | PR | HER2 | TNBC |
|---|---|---|---|---|
| Positive | 154 | 141 | 7 | 164 |
| Negative | 125 | 138 | 272 | 115 |

The inventors further found that the data shown in FIG. 5 and Table 1 correlate well with empirical data as well as with data obtained from PAM50 subtyping where TNBC typically correlates (to about 80%) with basal type breast cancer. Here, the inventors trained a 5-way classifier using PAM50 calls in TCGA BRCA cohorts, and then used robust averaging to ensure that it properly applies to the data sets obtained. As shown in Table 2, a PAM50 analysis provided 130 hits for Luminal A, 88 hits for basal, 60 hits for Luminal B, and 1 hit for Her2 enriched. The basal subtype is overrepresented (about 32%) compared to a randomized breast cancer population (10-20%). Table 3 shows overlap between TNBC (by inferred hormone status) and basal subtype (by PAM50 subtyper). The association analysis between predicted basal type in the PAM50 calculation and TNBC using contemplated methods herein had a p-value of <1.05e⁻⁴³ (using Fisher's exact test). It should be appreciated that the probability of achieving such strong association by chance is extremely small, indicating that the TNBC subgroup has been correctly identified in this cohort. In other words, it should be appreciated that RNAseq data may be effectively used to identify TNBC samples from a group of breast cancer samples.

TABLE 2

| Predicted PAM50 Subtype | Count |
|---|---|
| Luminal A | 130 |
| Basal | 88 |
| Luminal B | 60 |
| Her2-enriched | 1 |

TABLE 3

Predicted PAM50 Basal

| | | False | True |
|---|---|---|---|
| Inferred TNBC | False | 162 | 2 |
| status | True | 29 | 86 |

Consequently, the inventors further contemplate that a relatively large number of cancer tissue samples and the transcriptomics data (preferably filtered with threshold values by true positive and/or false negative values) are used to build and train an intrinsic subtype predictor for subtyping the cancer. Preferably the intrinsic subtype predictor can be built and trained using any machine learning system and/or algorithms. For example, suitable machine learning processes may read all relevant or selected omics data across all time points and biopsy location and perform training and validation splitting, data and metadata transformations, and then write those data to various formats required by disparate machine learning software packages. Suitable machine learning processes include glmnet lasso, glmnet ridge regression, glmnet elastic nets, NMFpredictor, WEKA SMO, WEKA j48 trees, WEKA hyperpipes, WEKA random forests, WEKA naive Bayes, WEKA JRip rules, etc. Exemplary machine learning processes are disclosed in WO 2014/059036 or WO 2014/193982, which are incorporated by references herein. Moreover, mutational data may be employed to further refine the gene set or to associate mutations with one or more expression levels.

The inventors further found that the machine learning process to classify and/or characterize the cancer tissue using transcriptomics data can be more efficiently and/or effectively performed when the transcriptomics data are clustered into a plurality of clusters (e.g., based on the level of up- or down-regulation, based on the absolute expression level, based on the associated changes with other genes, based on the associated changes with specific types of cancer tissue, etc.). Thus, the number of clusters of transcriptomics may vary, and the number of genes in each cluster may vary as well. For example, the number of clusters may be at least 3 clusters, at least 5 clusters, at least 10 clusters, at least 15 clusters, at least 20 clusters, and the number of genes in each cluster may range between 10-10,000 genes, between 10-1000 genes, between 10-100 genes, etc.

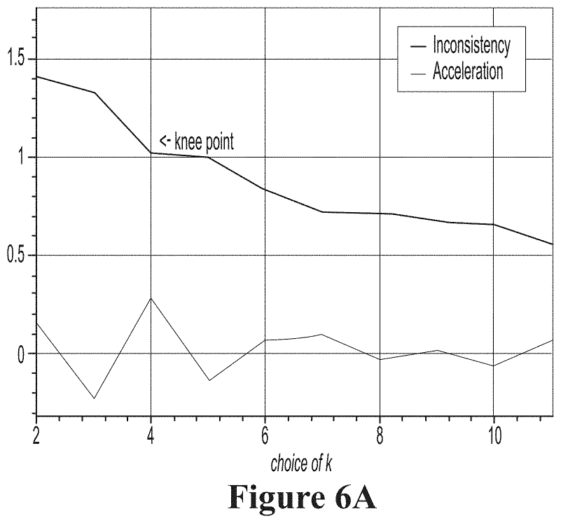

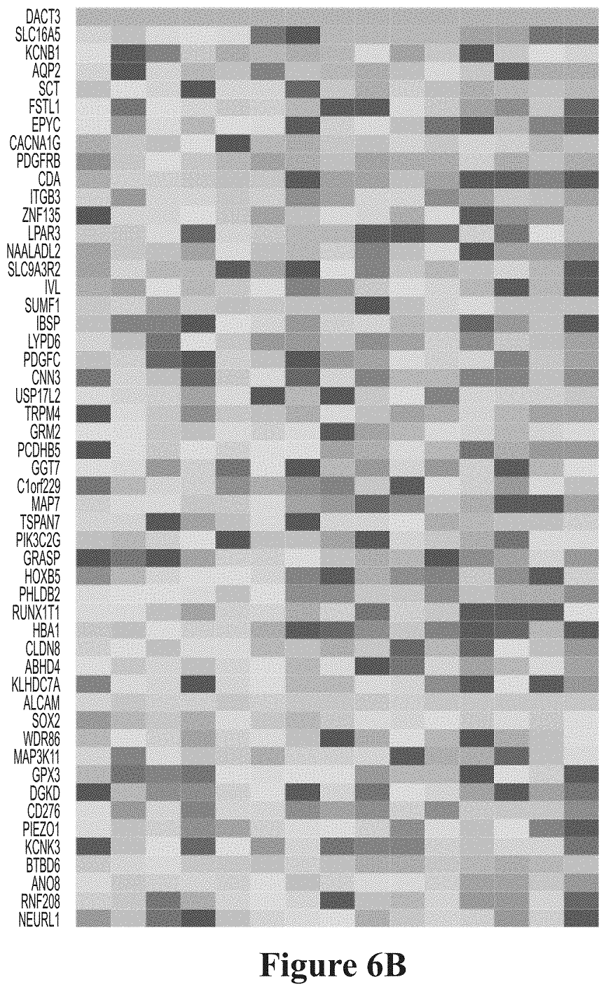

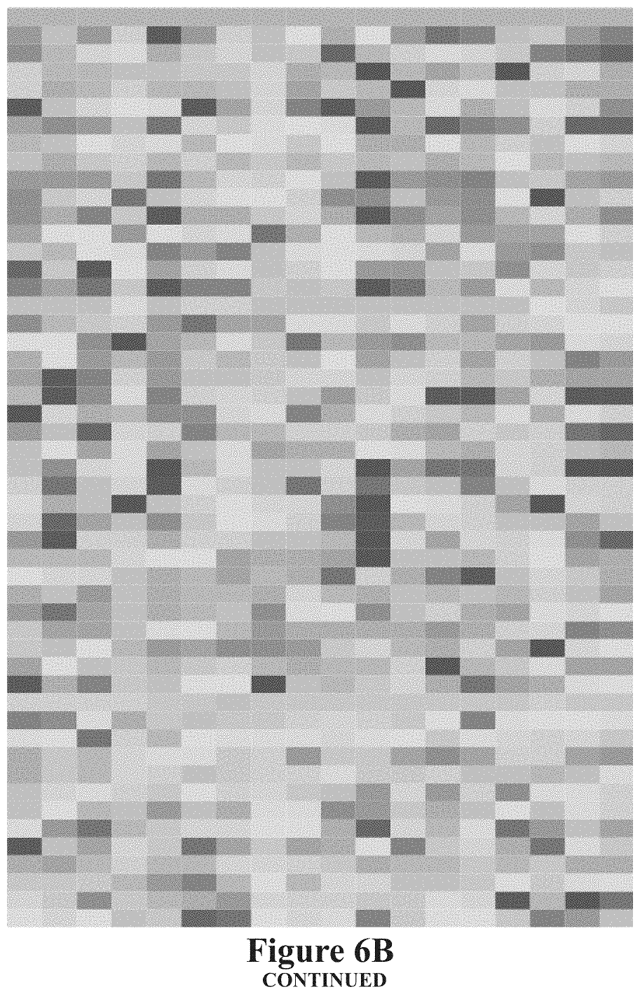

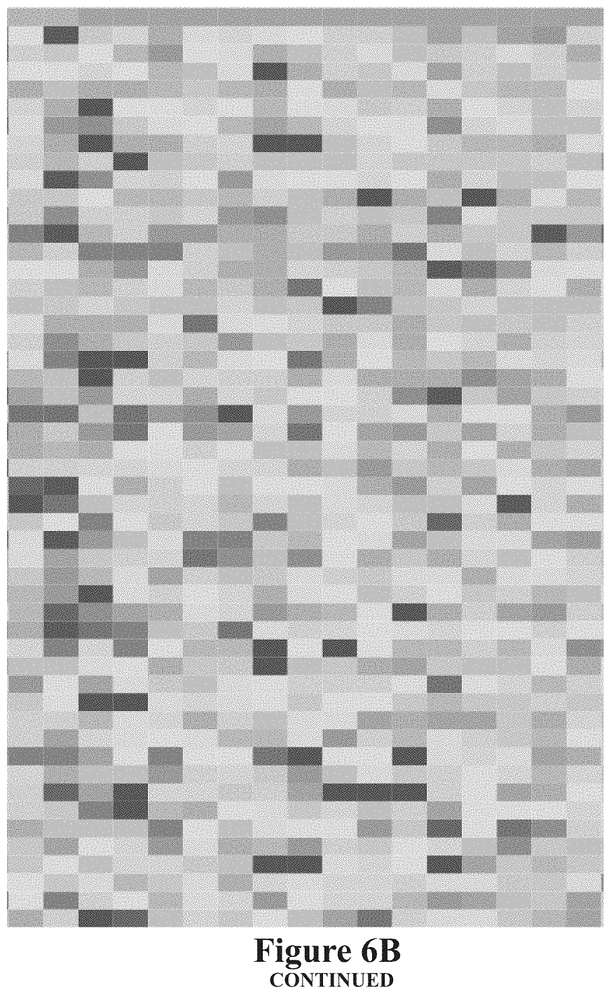

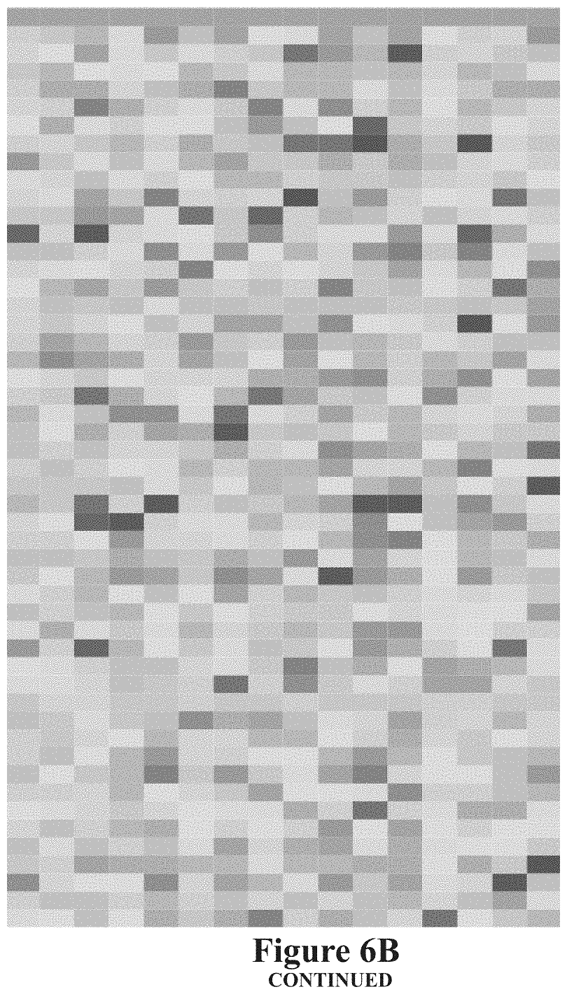

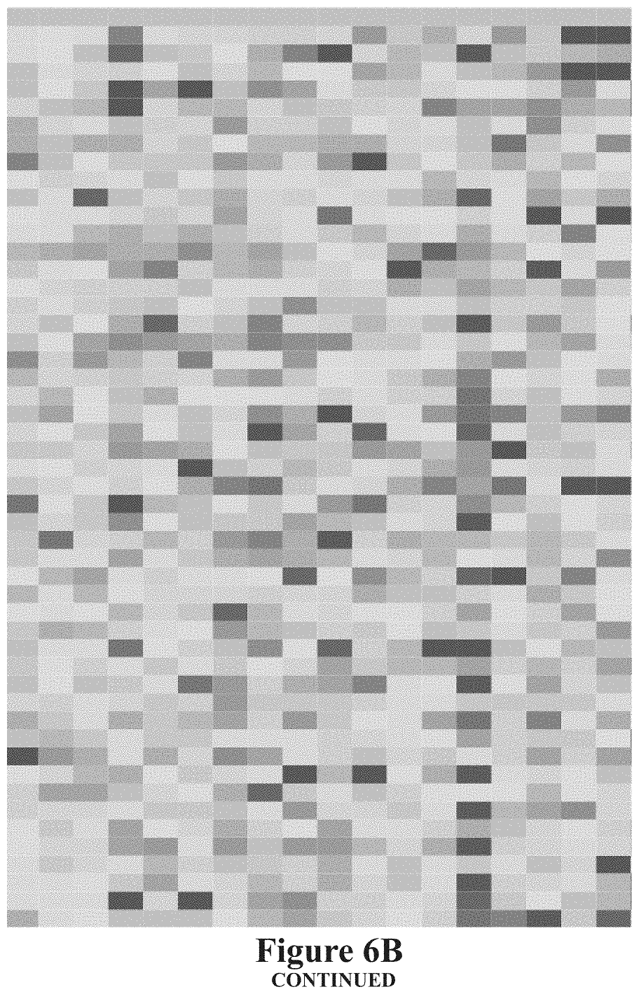

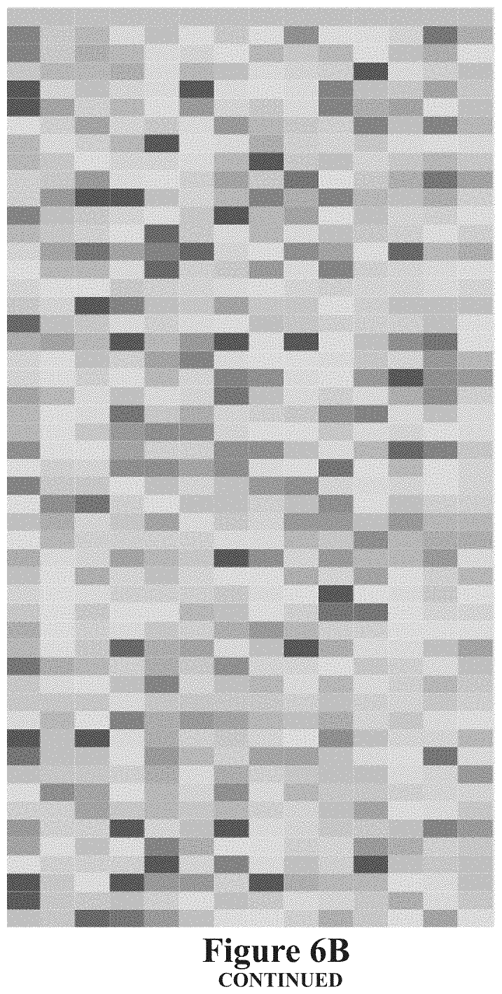

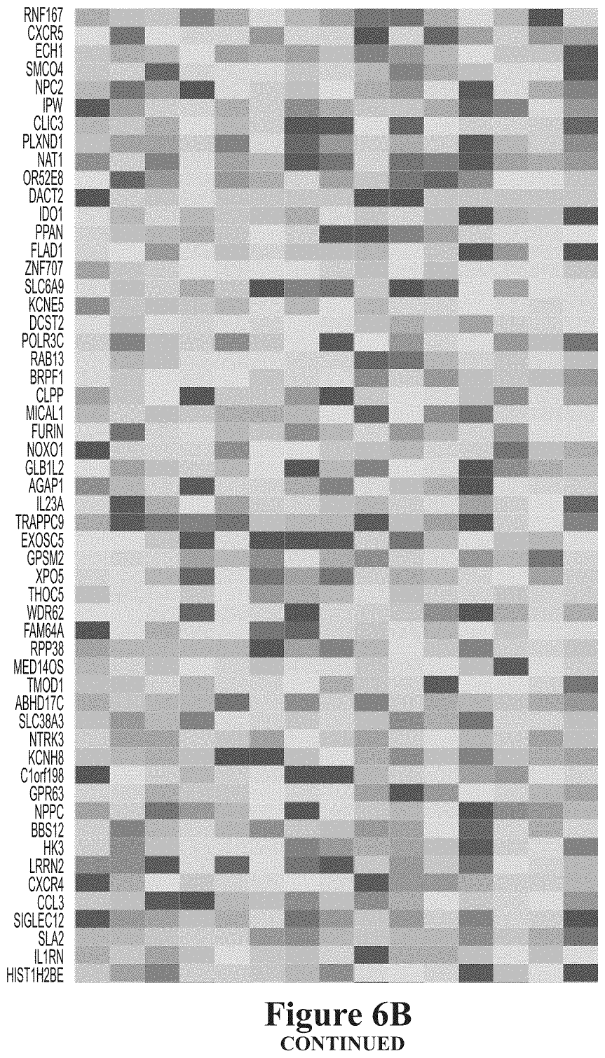

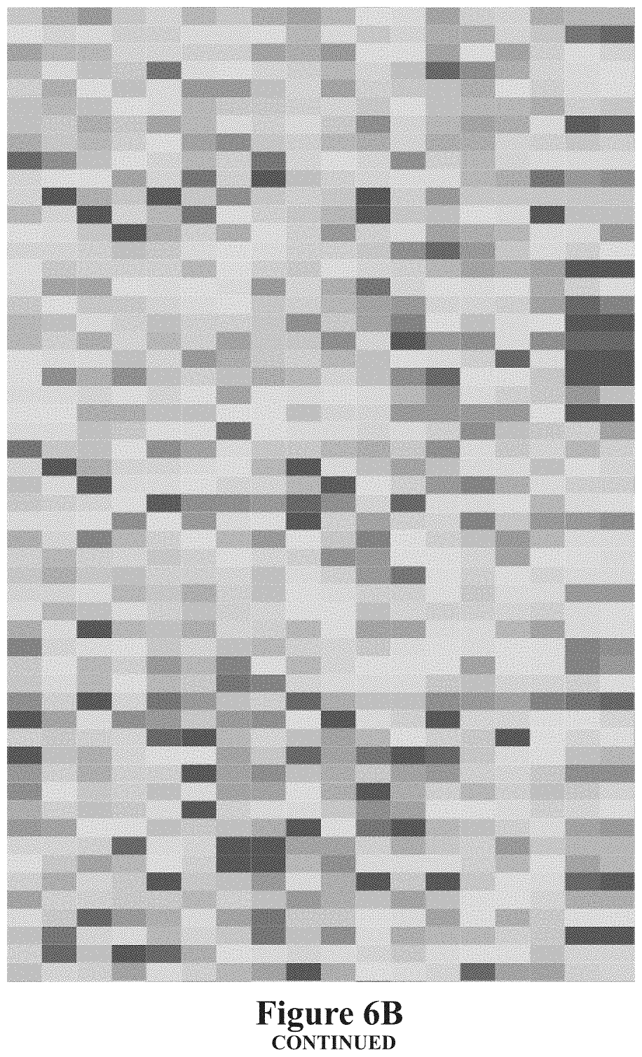

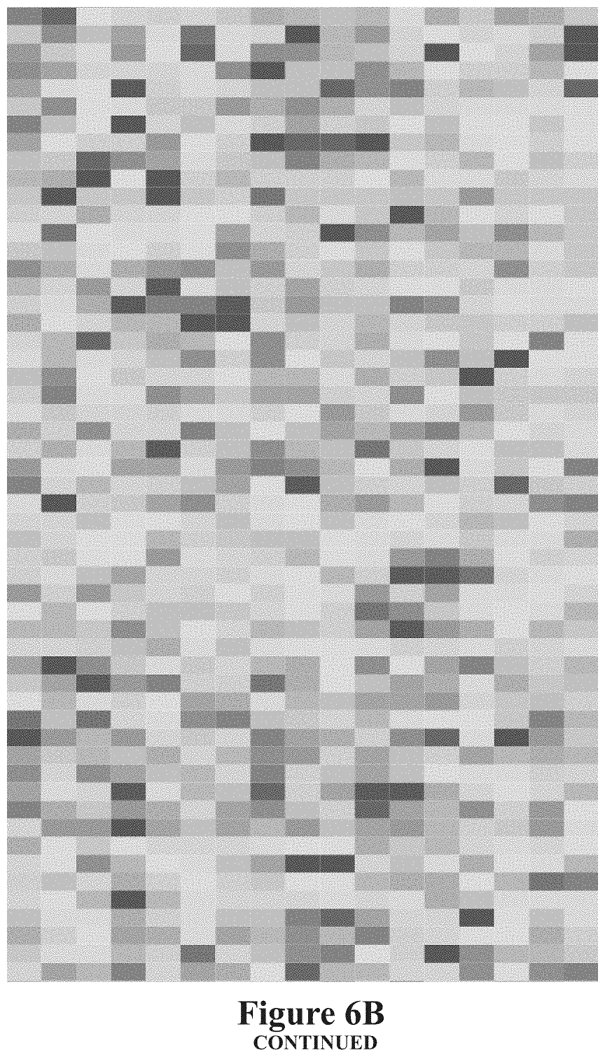

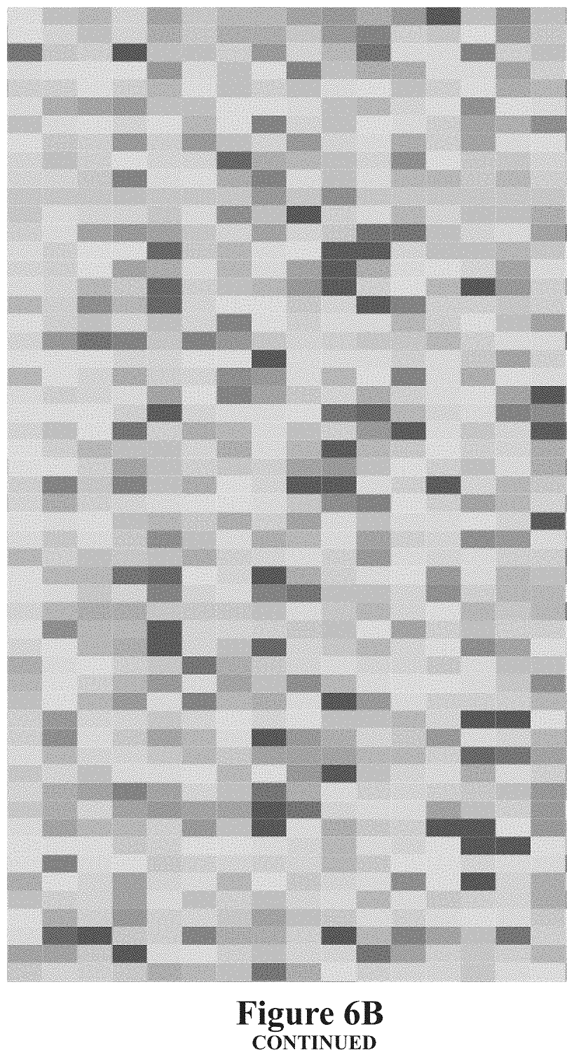

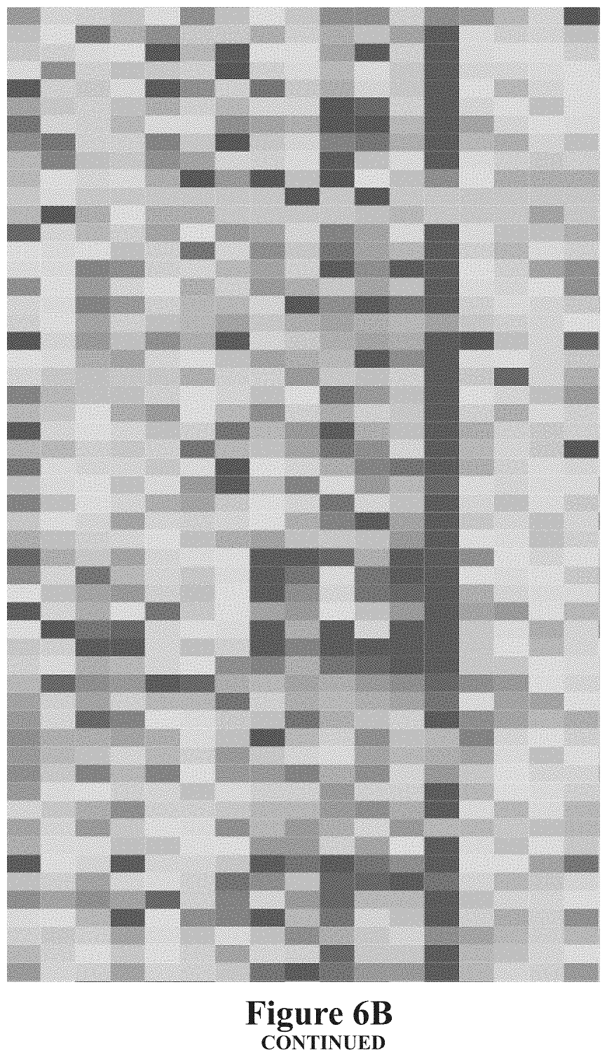

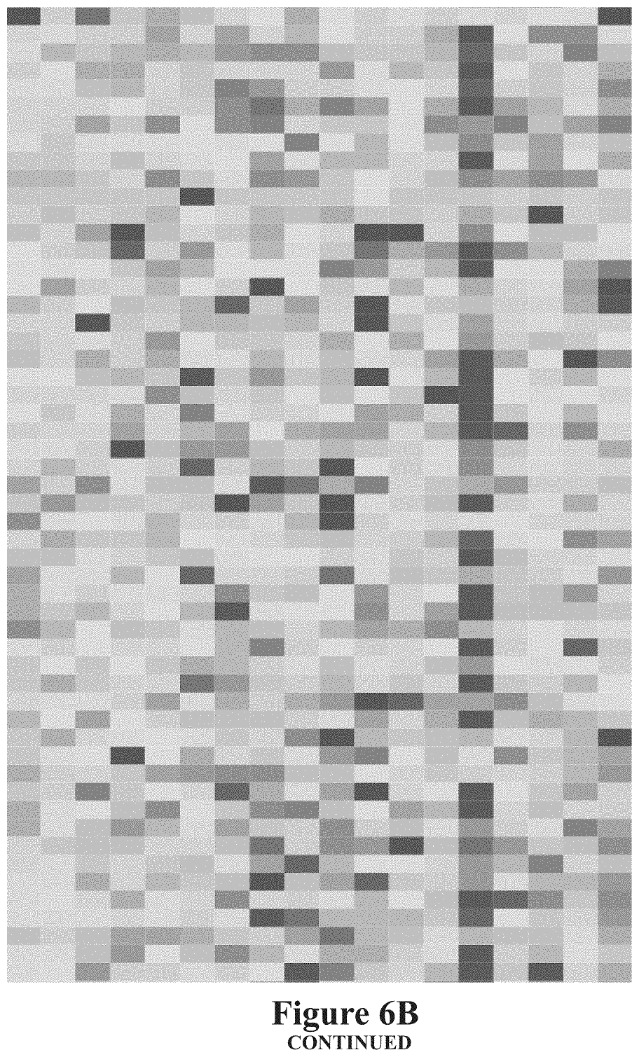

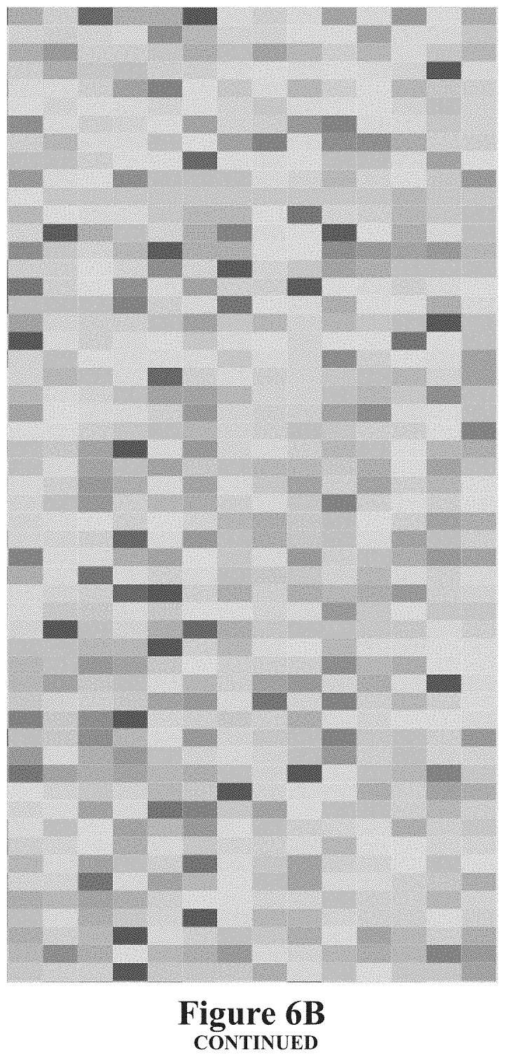

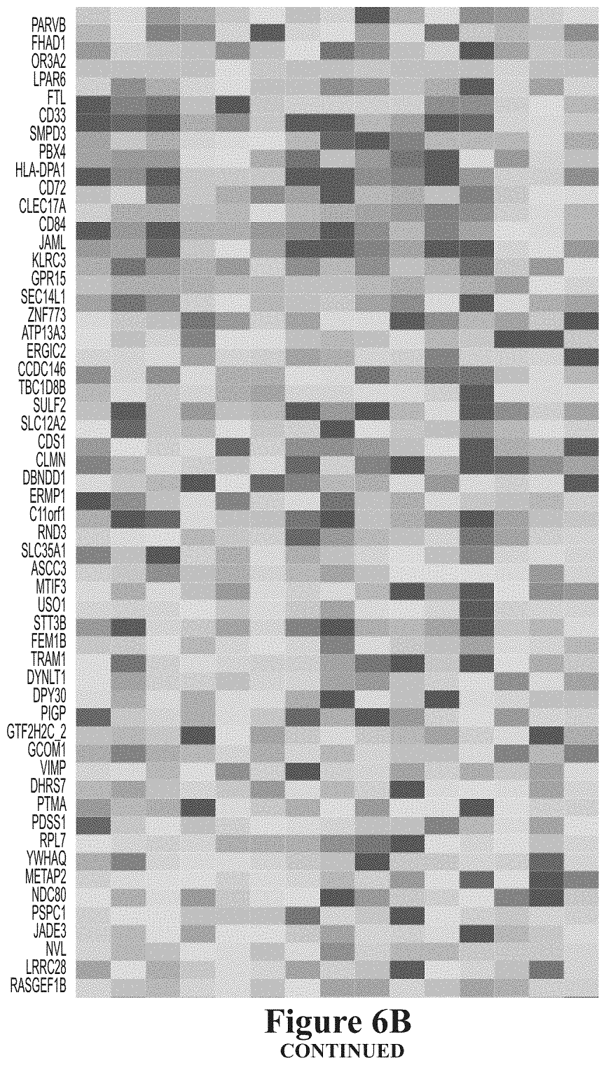

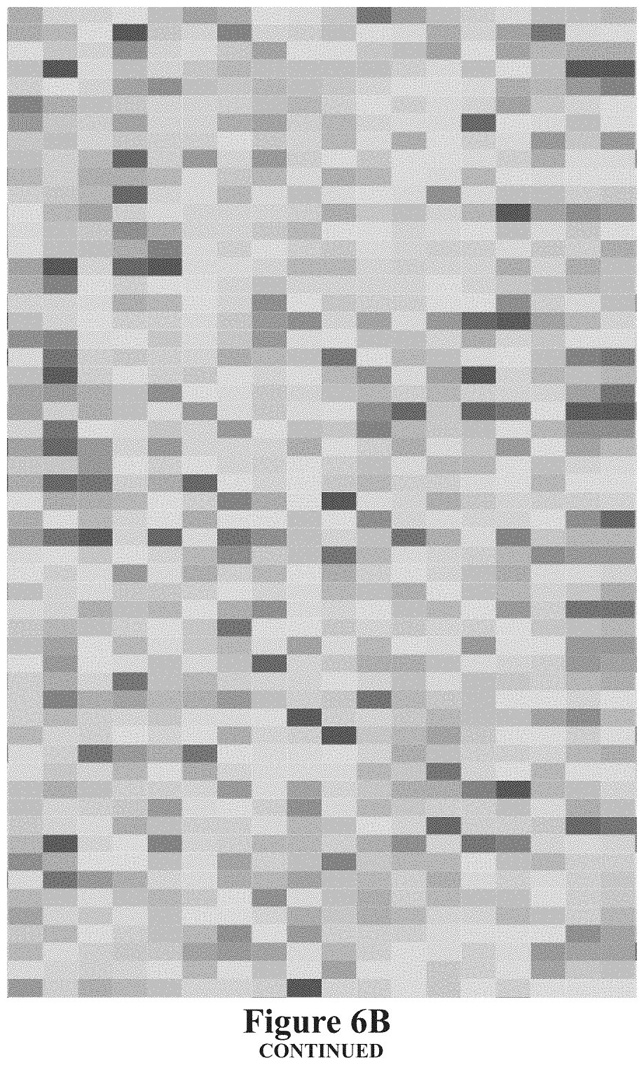

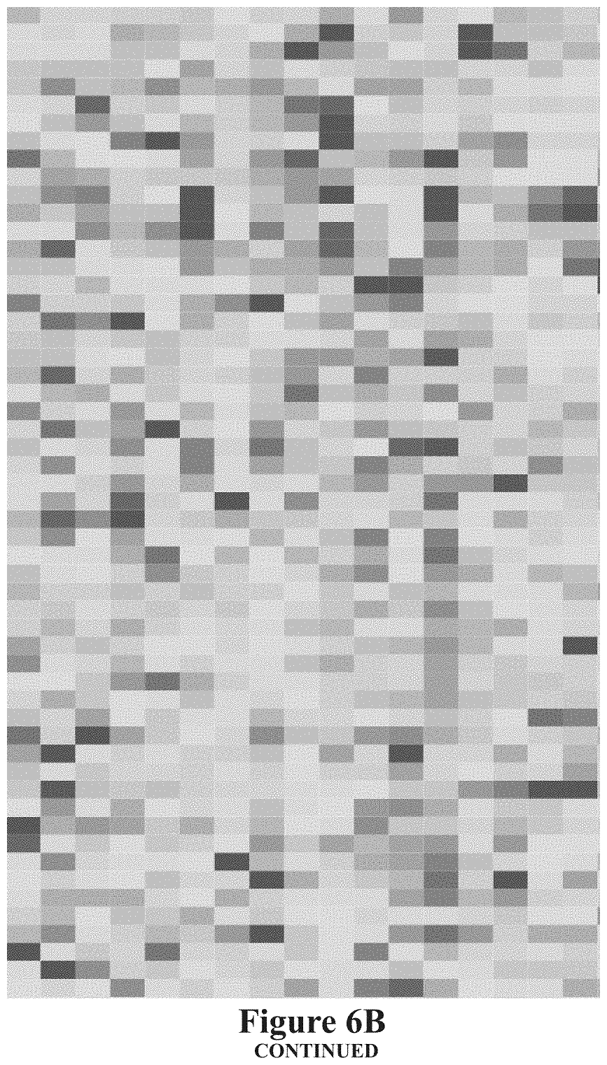

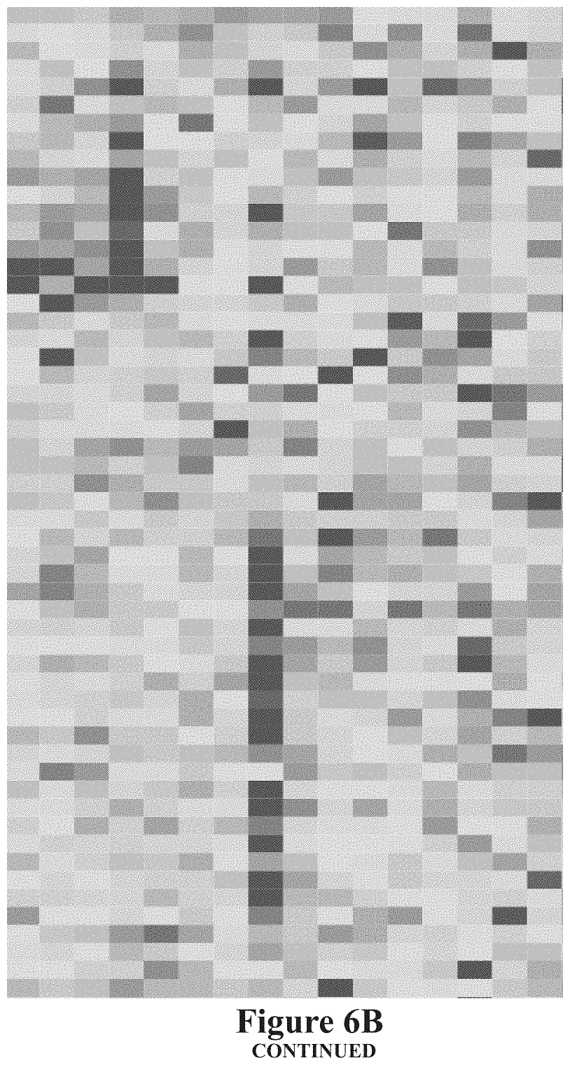

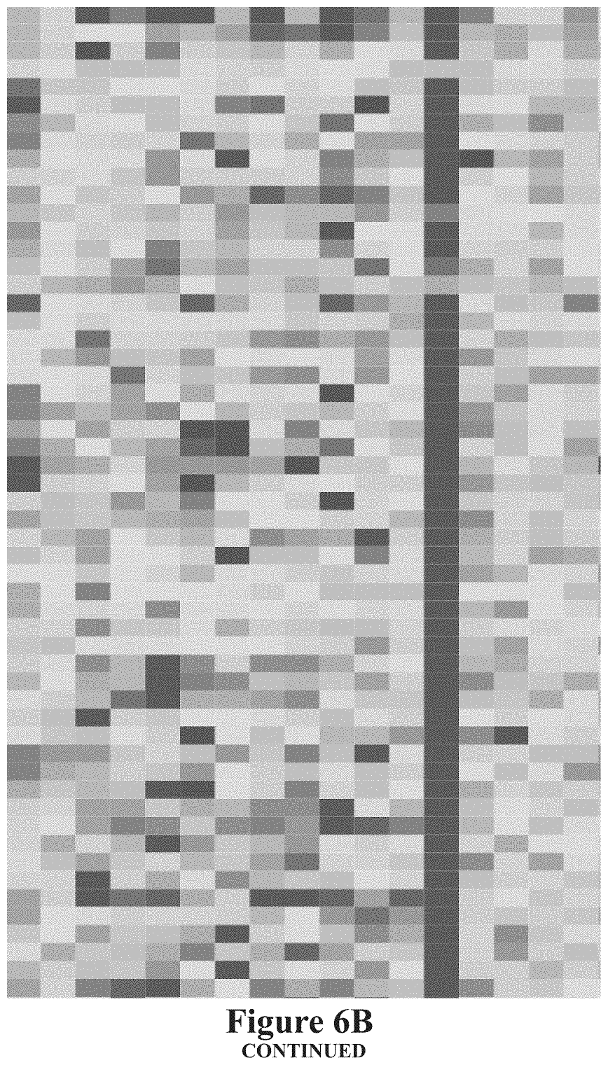

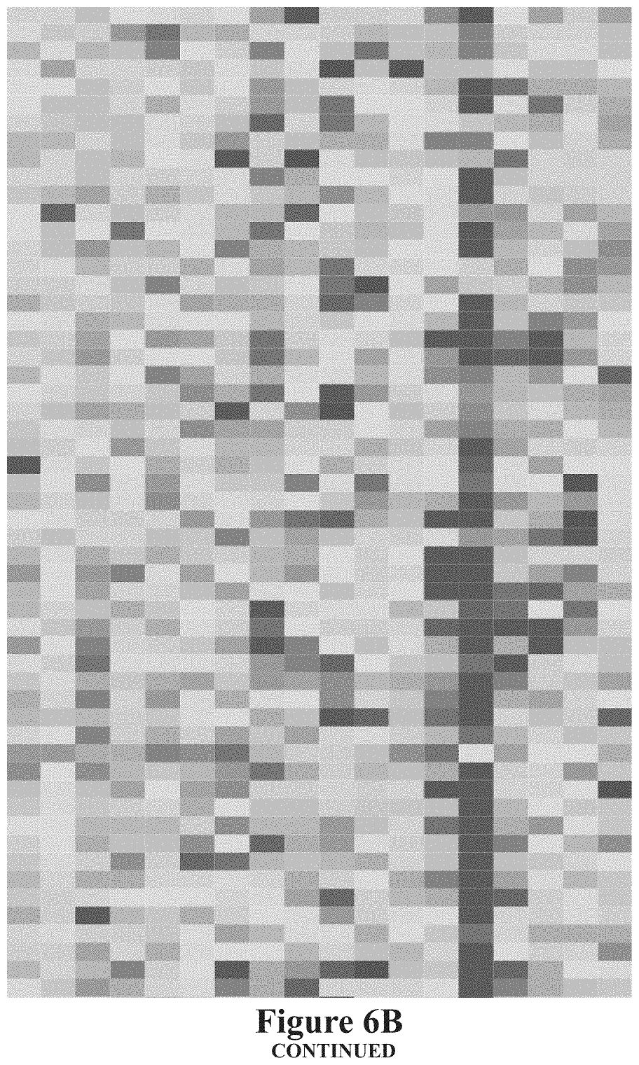

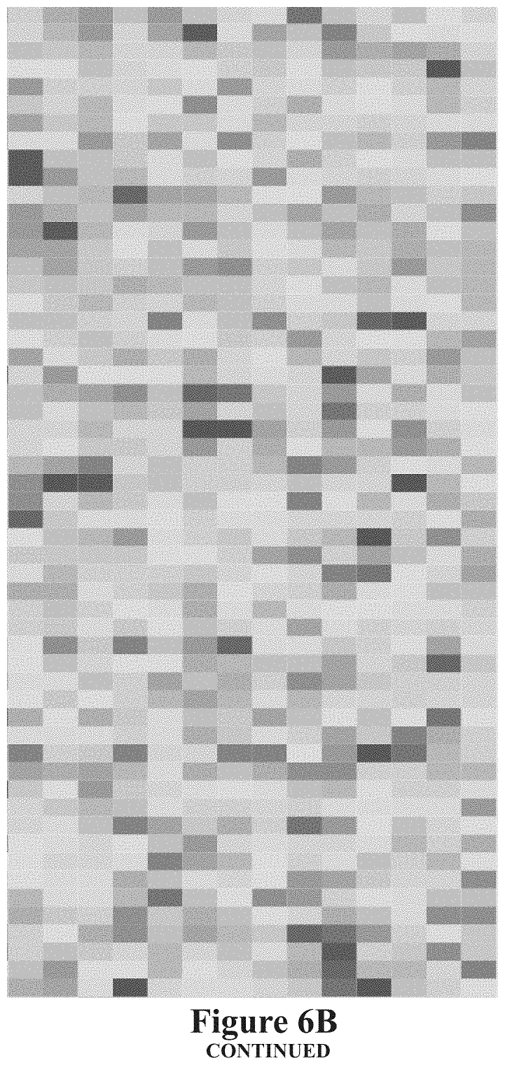

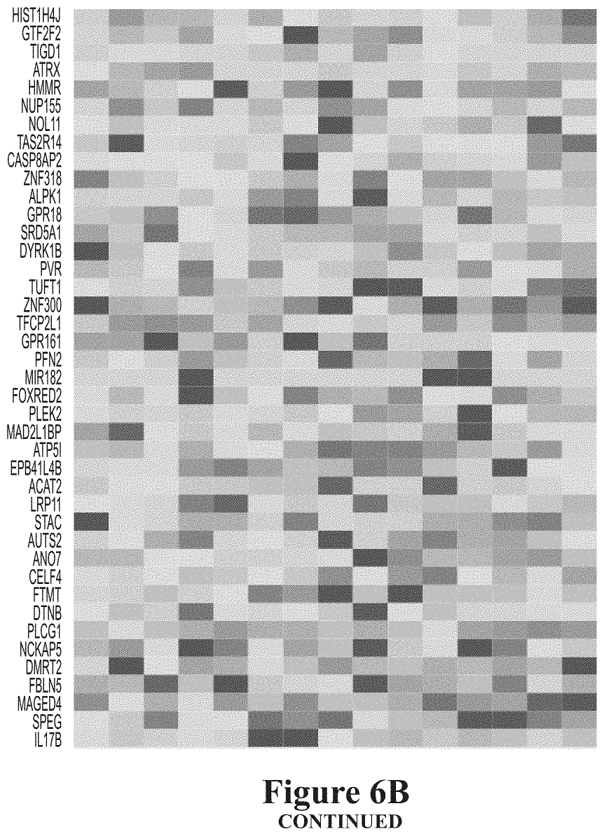

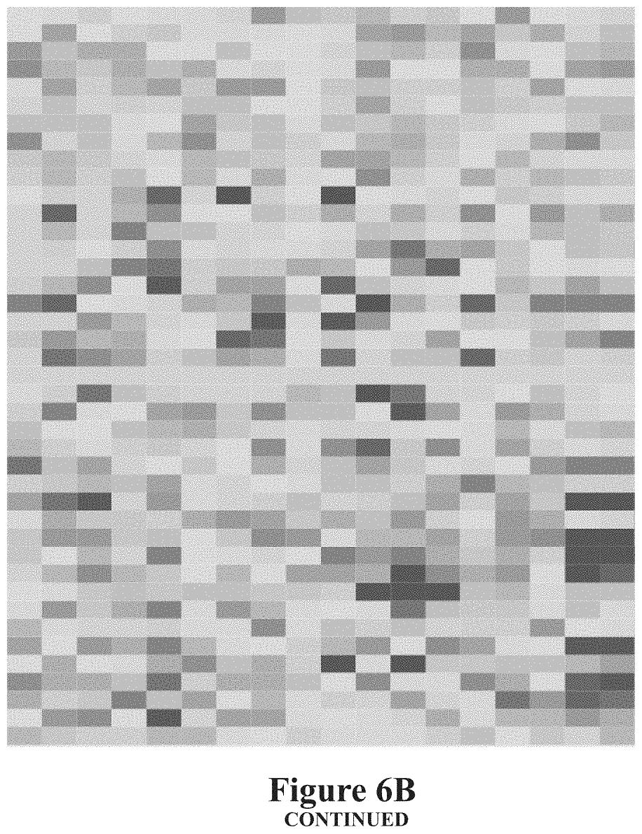

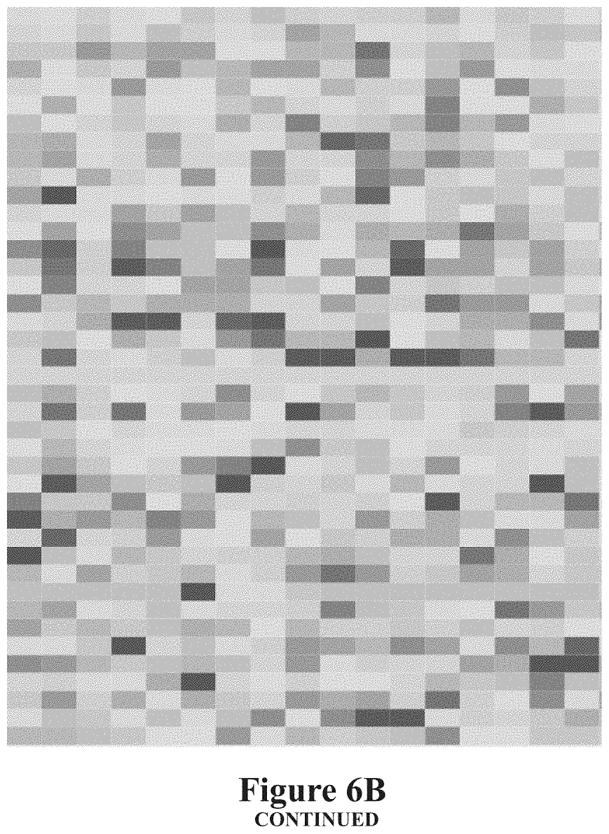

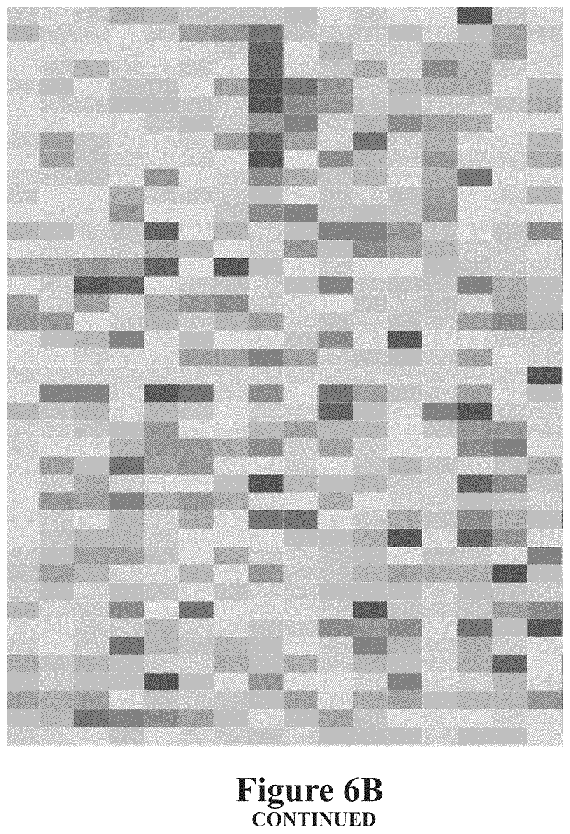

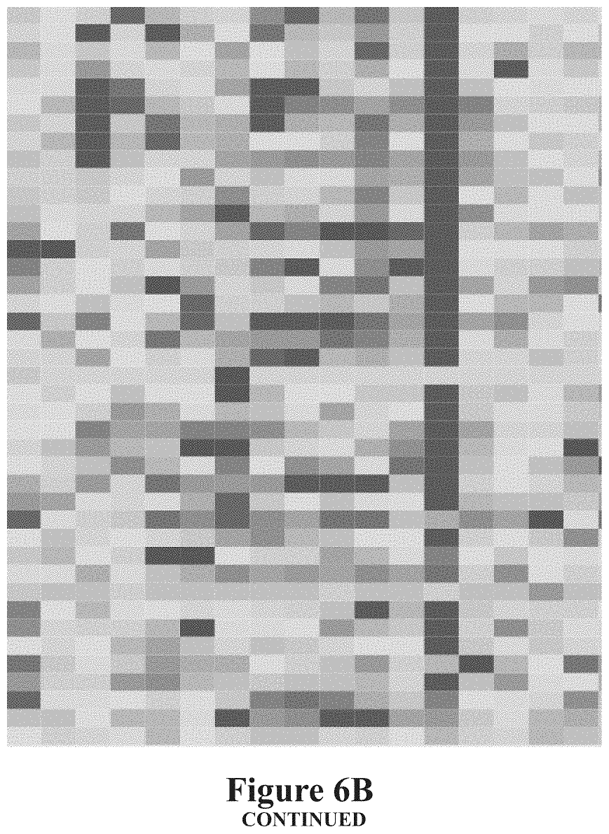

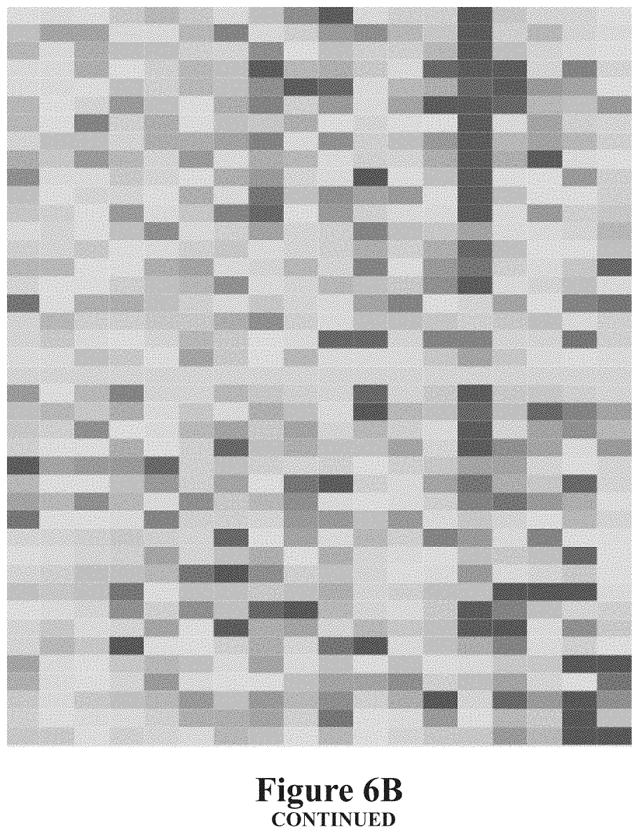

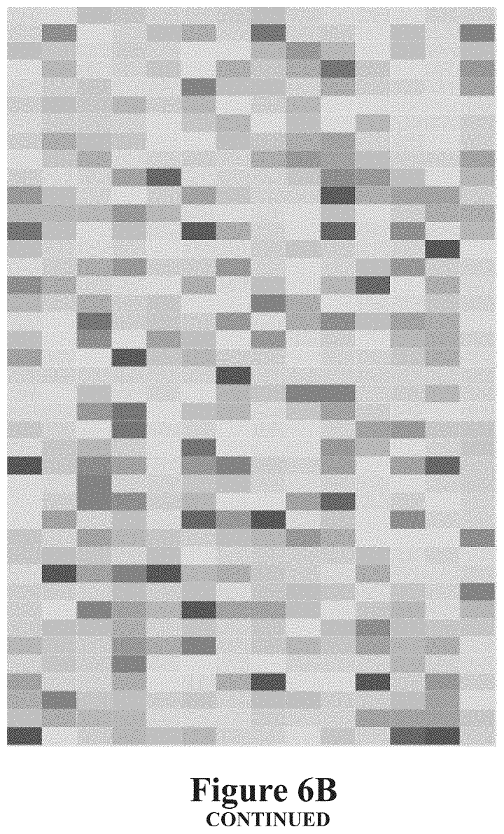

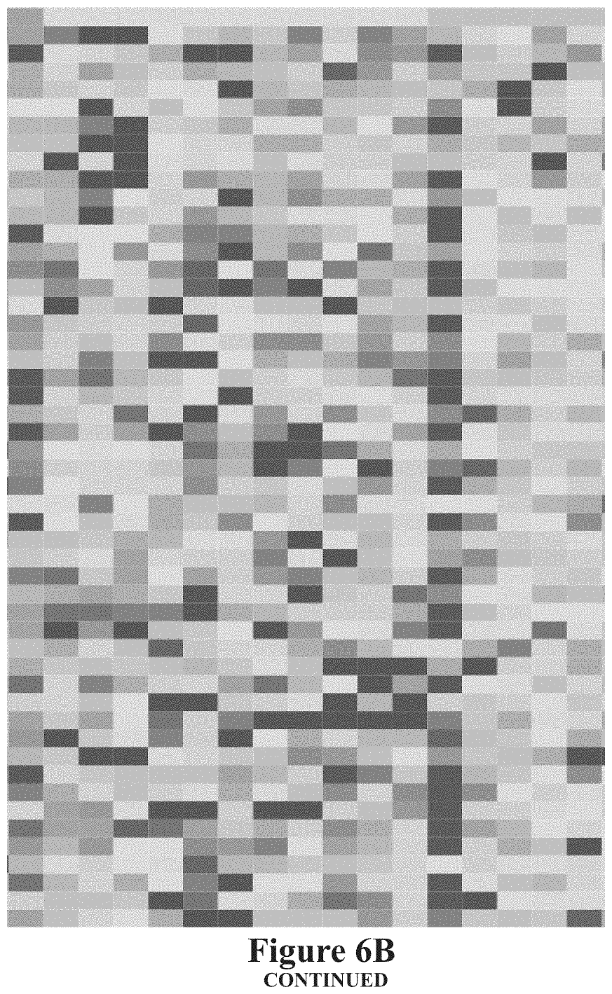

Consequently, the inventors contemplate that an optimal number of clusters can be selected to increase the efficiency of the machine learning for characterizing and/or classifying the cancer tissues. Preferably, the optimal or appropriate number of clusters can be selected using a knee point analysis identifying a point with the largest acceleration with decreased inconsistency. For example, the inventors further subject all identified TNBC samples to an analysis to identify subtypes independent of any classifier. The inventor first defined a set of clusters that was considered gold-standard but included too many genes suitable for diagnostic use. More specifically, the initially selected genes were highly differentially expressed (i.e., most variable genes) within the TNBC group. This group of genes included approximately 10,000 genes. To identify an appropriate number of clusters, a knee point analysis was performed on a restricted set of data (here 115 patient data using the 10,000 most variant genes). As can be taken from FIG. 6A, the largest acceleration (decrease in inconsistency) was observed at k=4 (cluster numbers of 4) in a K-means clustering.

While there can be 10,000 mostly variable genes related to the breast cancer classification, such number of genes are often too many for further analysis, especially to visualize the clusters. Thus, in FIG. 6B, instead of entire 10,000 genes, every 50^{th} gene can be plotted for each cluster for visualization of the cluster as a heatmap of expression values for 200 such randomly selected genes from the full 10k list of genes (most variably expressed genes) that are shown as a row and are grouped into 4 clusters (as shown in 4 discontinuous bar at the top of the heat map). The genes depicted in the heatmap includes IL17B, SPEG, MAGED4, FBLN5, DMRT2, NCKAP5, PLCG1, DTNB, FTMT, CELF4, ANO7, AUTS2, STAC, LRP11, ACAT2, EPB41L4B, ATP5I, MAD2L1BP, PLEK2, FOXRED2, MIR182, PFN2, GPR161, TFCP2L1, ZNF300, TUFT1, PVR, DYRK1B, SRD5A1, GPR18, ALPK1, ZNF318, CASP8AP2, TAS2R14, NOL11, NUP155, HMMR, ATRX, TIGD1, GTF2F2, HIST1H4J, RASGEF1B, LRRC28, NVL, JADE3, PSPC1, NDC80, METAP2, YWHAQ, RPL7, PDSS1, PTMA, DHRS7, VIMP, GCOM1, GTF2H2C_2, PIGP, DPY30, DYNLT1, TRAM1, FEM1B, STT3B, USO1, MTIF3, ASCC3, SLC35A1, RND3, C11orf1, ERMP1, DBNDD1, CLMN, CDS1, SLC12A2, SULF2, TBC1D8B, CCDC146, ERGIC2, ATP13A3, ZNF773, SEC14L1, GPR15, KLRC3, JAML, CD84, CLEC17A, CD72, HLA-DPA1, PBX4, SMPD3, CD33, FTL, LPAR6, OR3A2, FHAD1, PARVB, HIST1H2BE, IL1RN, SLA2, SIGLEC12, CCL3, CXCR4, LRRN2, HK3, BBS12, NPPC, GPR63, C1orf198, KCNH8, NTRK3, SLC38A3, ABHD17C, TMOD1, MED14OS, RPP38, FAM64A, WDR62, THOC5, XPO5, GPSM2, EXOSC5, TRAPPC9, IL23A, AGAP1, GLB1L2, NOXO1, FURIN, MICAL1, CLPP, BRPF1, RAB13, POLR3C, DCST2, KCNE5, SLC6A9, ZNF707, FLAD1, PPAN, IDO1, DACT2, OR52E8, NAT1, PLXND1, CLIC3, IPW, NPC2, SMCO4, ECH1, CXCR5, RNF167, NEURL1, RNF208, ANO8, BTBD6, KCNK3, PIEZO1, CD276, DGKD, GPX3, MAP3K11, WDR86, SOX2, ALCAM, KLHDC7A, ABHD4, CLDN8, HBA1, RUNX1T1, PHLDB2, HOXB5, GRASP, PIK3C2G, TSPAN7, MAP7, C1orf229, GGT7, PCDHB5, GRM2, TRPM4, USP17L2, CNN3, PDGFC, LYPD6, IBSP, SUMF1, IVL, SLC9A3R2, NAALADL2, LPAR3, ZNF135, ITGB3, CDA, PDGFRB, CACNA1G, EPYC, FSTL1, SCT, AQP2, KCNB1, SLC16A5, DACT3. Such set of 4 subgroups establishes a gold standard for further analysis.

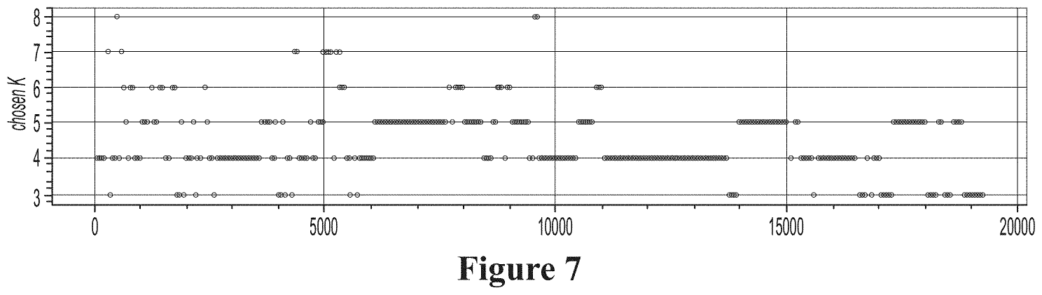

FIG. 7 shows an exemplary comparison of data consistency in each cluster as a function of size of data sets. Gene set sizes ranging from 50 to 19250 (x-axis) were tested for optimal K between 3 and 10 (y-axis), and Counts for number of times each K was selected using varying gene set sizes. As shown in Table 4, K=4 was most consistently (or frequently) selected as fitting the TNBC subset of the GeparSepto data the best, in any sizes of data sets.

TABLE 4

| Chosen K | # times selected |
|---|---|
| 4 | 173 |
| 5 | 127 |
| 3 | 45 |
| 6 | 28 |
| 8 | 2 |

While a cluster size of 4 was so determined the best clustering in the example depicted in FIGS. 6A-B, the number of genes for transcriptomics data is still undesirably large. In a preferred embodiment, the number of genes per cluster can be reduced until the number reaches to the optimal number of genes per cluster (e.g., less than 100 genes per cluster, less than 50 genes per cluster, less than 30 genes per cluster, etc.). While any suitable methods to reduce the number of genes per cluster are contemplated, preferred method includes use of a recursive feature elimination process to reduce the number of genes necessary to obtain almost the same clustering. More specifically, in a first step of the recursive feature elimination, 4 one-vs-rest classifiers (one for each cluster, 1 versus 2-4, then 2 versus 1 and 3-4, etc.) can be trained. The gene weights in each classifier are then inspected to obtain respective lists of genes most useful for defining the classes. Reduction of the gene set is then implemented by only keeping a fraction (e.g., 20%, 25%, 30%, 40%, 50%) of the genes from each classifier, and by merging all of the reduced lists into one list (e.g., with approximately half the features of the original dataset). Clustering and culling is repeated using the same process on the reduced set, and if homogeneity (i.e., agreement of samples co-clustering) was high enough, the reduced feature set is the new dataset. It should be appreciated that this process of building 4-way classifiers, dropping low-coefficient genes, and re-clustering, can be repeated until the homogeneity drops too low (e.g., below 60%, or below 50% agreement with the original ‘gold-standard’ clusters). Thus, the clustering and culling process using recursive feature elimination may be repeated once, preferably at least twice, five times, or even ten times until the reduced transcriptomics data is less than 60%, less than 55%, less than 50%, less than 45%, less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% of the total or original transcriptomic data of the cancer tissue in number or by volume. Remarkably, using this approach the inventor could reduce the original set of 10,000 gene expression data to only 79 gene expression data that essentially provided the same clustering.

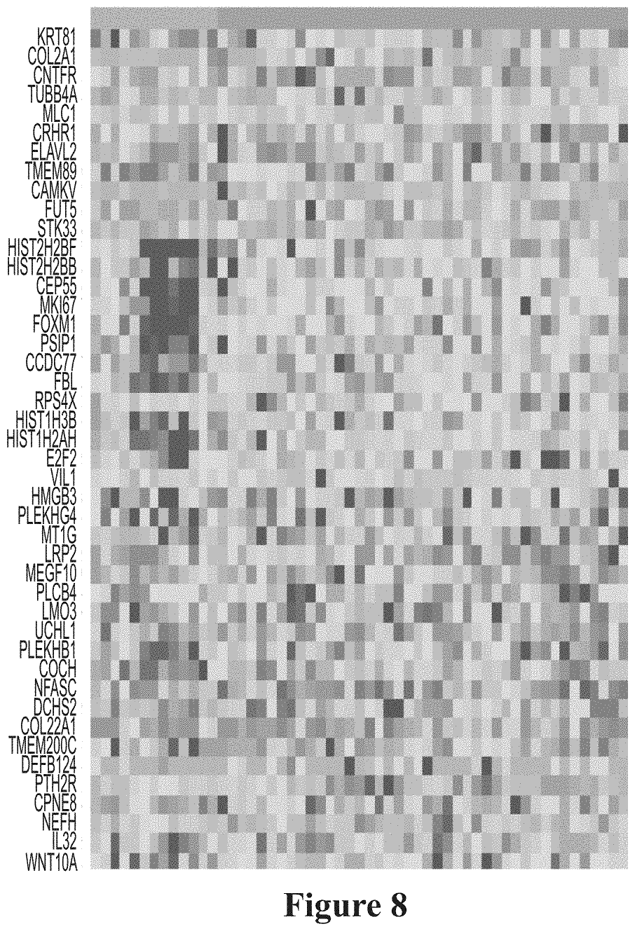

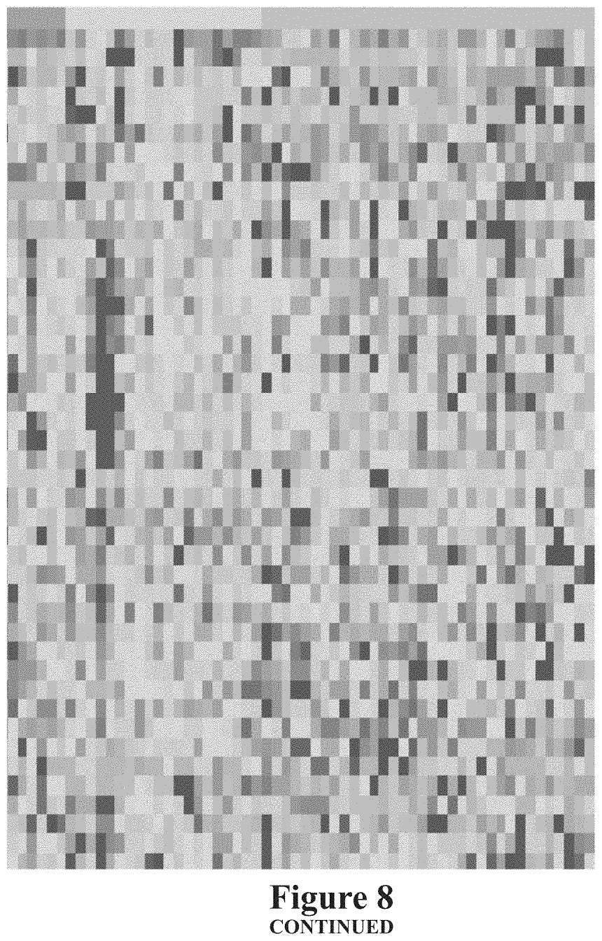

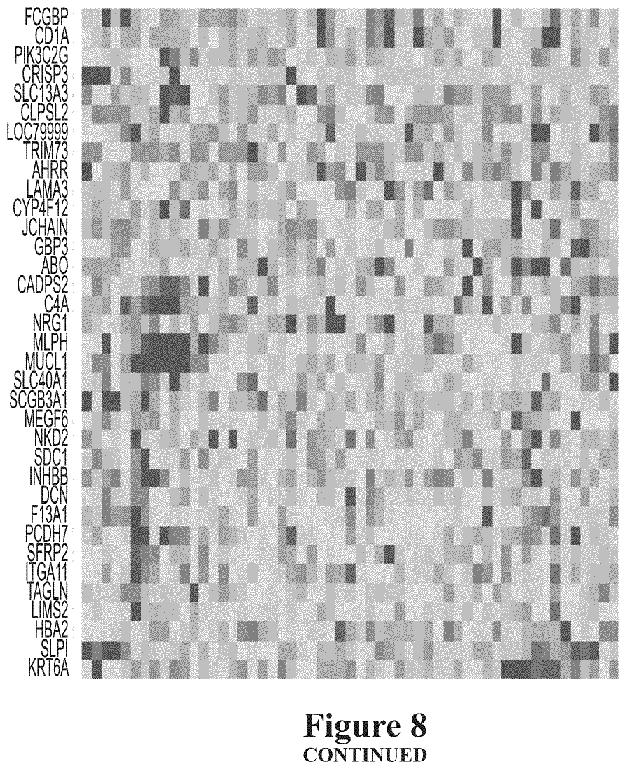

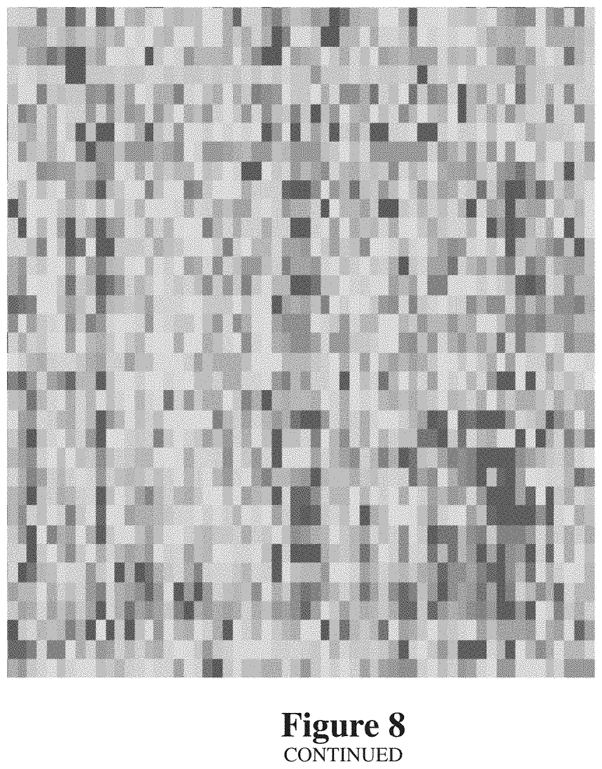

FIG. 8 schematically illustrates a heat map with 4 clusters using the reduced gene set prepared as described above. In this example, and for TNBC, the reduced gene set includes the following genes: KRT81, COL22A1, CNTFR, TUBB4A, MLC1, CRHR1, ELAVL2, TMEM89, CAMKV, FUT5, STK33, HIST2H2BF, HIST3H2BB, CEP55, MKI67, FOXM1, PSIP1, CCDC77, FBL, RPS4X, HIST1H3B, HIST1H2AH, E2F2, VIL1, HMGB3, PLEKHG4, MT1G, LRP2, MEGF10, PLCB4, LMO3, UCHL1, PLEKHB1, COCH, NFASC, DCHS2, COL22A1, TMEM200C, DEFB124, PTH2R, CPNE8, NEFH, IL32, WNT10A, FCGBP, CD1A, PIK3C2G, CRISP3, SLC13A3, CLPSL2, LOC79999, TRIM73, AHRR, LAMA3, CYP4F12, JCHAIN, GBP3, ABO, CADPS2, C4A, NRG1, MLPH, MUCL1, SLC40A1, SCGB3A1, MEGF6, NKD2, SDC1, INHBB, DCN, F13A1, PCDH7, SFRP2, ITGA11, TAGLN, LIMS2, HBA2, SLPI, and KRT6A. The inventors further queried the gene list against six available data bases (NCINature_2016, BioCarta_2016, GO_Biological_Process_2015, GO_Molecular_Function_2015, KEGG_2016, and WikiPathways_2016). Table 5 shows a subset of the databases and gene sets that are significantly associated with reduced gene sets in 4 clusters (adjusted p value <0.1).

TABLE 5

| | | Adjusted | | |
|---|---|---|---|---|
| Term | Overlap | p value | Genes | Database |
| Beta1 integrin cell | 4/66 | 0.004048 | COL2A1; ITGA11; | NCI- |
| surface interactions_Homo . . . | | | LAMA3; F13A1 | Nature_2016 |
| Systemic lupus | 5/135 | 0.014516 | C4A; HIST1H2AH; | KEGG_2016 |
| erythematosus_Homo sapiens_hsa0 . . . | | | HIST3H2BB; HIST1H3B; | |
| | | | HIST2H2BF | |
| ECM-receptor | 4/82 | 0.014516 | COL2A1; ITGA11; | KEGG_2016 |
| interaction_Homo sapiens_hsa04512 | | | LAMA3; SDC1 | |
| Wnt signaling | 4/142 | 0.075132 | WNT10A; SFRP2; | KEGG_2016 |
| pathway_Homo sapiens_hsa04310 | | | PLCB4; NKD2 | |

It is contemplated that the reduced gene sets clustered in an optimal number of clusters (e.g., k=4) can substantially increase the efficiency and speed of the transcriptomics analysis to classify and/or characterize the cancer tissue as the amount of data to be processed can be at least 10 times, at least 50 times, at least 100 times smaller than the whole transcriptomics analysis. Further, such reduced gene sets in each cluster may reduce the false positive data and/or false negative data due to the high variance of the transcriptomics data among tissues such that the accuracy of the analysis can be substantially increased. Preferably, subtyping is unsupervised and based on recursive feature elimination of a large set of genes with highest variability in gene expression.

In addition, the results of such clustering of cancer tissues can be used as an input into pathway analysis algorithms to identify affected and/or targetable pathways and/or intrinsic properties of the tumor tissue or cells. In some embodiments, the transcriptomics data of selected genes (in each cluster or one of the clusters) can be integrated into a pathway model (e.g., as a pathway element or a regulatory parameter to control or affect the pathway element, etc.) to generate a modified pathway of cancer tissue to determine any differential pathway characteristic of the cancer tissue. While any suitable methods of analyzing pathway characteristics of cells are contemplated, a preferred method uses PARADIGM (Pathway Recognition Algorithm using Data Integration on Genomic Models), which is a genomic analysis tool described in WO2011/139345 and WO/2013/062505 and uses a probabilistic graphical model to integrate multiple genomic data types on curated pathway databases.

Further, it is also contemplated that classification and/or characterization of the cancer tissue may be advantageously associated (preferably via machine learning) with a desired treatment or predictive parameter, and/or improved by use of supervised learning. For example, a specific subtype as presented herein may be associated with treatment response to nab-paclitaxel, optionally followed by epirubicin plus cyclophosphamide. Likewise, a specific subtype as presented herein may be associated with the overall survival rate or a disease free or progression free survival time. As will be readily appreciated, the results of such clustering can be used to stratify breast cancer patient data, and/or used in supervised machine learning using various classifiers, and particularly drug response (e.g., NAB paclitaxel, optionally with epirubicin/cyclophosphamide), overall survival prediction, or prediction of disease free survival or progression free survival.

In some embodiments, such association with drug sensitivity, predicted treatment response, overall survival rate or a disease free or progression free survival time can be further used to generate and/or determine a treatment regimen. For example, the predicted treatment response using nab-paclitaxel is highly positive, the treatment regimen to the patient can include nab-paclitaxel. In addition, the effect of nab-paclitaxel treatment to the tumor tissue can be simulated in a pathway analysis to determine any potential changes in the pathway activity in one or more selected genes in the cluster. In such scenario, a treatment targeting the one or more selected genes that are (potentially) changed by nab-paclitaxel treatment can be further selected as a treatment regimen followed by nab-paclitaxel treatment. As used here, a treatment targeting a gene refers a treatment targeting (e.g., binding, inhibiting the activity, enhancing the activity, etc.) a protein encoded by the gene, and/or a treatment inhibiting or enhancing the gene expression of the one or more genes in a transcriptional level, in a translational level, and/or in a post-translational modification level (e.g., phosphorylation, glycosylation, protein-protein binding, etc.). Such determined or generated treatment (regimen) can be further administered to the patient having the tumor in a dose and a schedule effective or sufficient to treat the tumor (e.g., to reduce the tumor size, to increase the immune response against the tumor, to increase the survival rate, etc.). As used herein, the term “administering” refers to both direct and indirect administration of the treatment regimens, drugs, therapies contemplated herein, where direct administration is typically performed by a health care professional (e.g., physician, nurse, etc.), while indirect administration typically includes a step of providing or making the compounds and compositions available to the health care professional for direct administration.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of processing omics data of a cancer tissue, comprising:
obtaining transcriptomic data of the cancer tissue, wherein the transcriptomics data is associated with protein expression level of a plurality of proteins in the cancer tissue, and wherein the plurality of proteins is associated with a phenotype of the cancer tissue;
stratifying the transcriptomics data into a subgroup of data, and clustering the subgroup of data; and
subjecting the clustered subgroup of data to recursive feature elimination to obtain reduced transcriptomic data.

2. The method of claim 1, wherein the cancer sample is a breast cancer sample, and in which the plurality of proteins includes at least one of an estrogen receptor, a progesterone receptor, and HER2.

3. The method of claim 1, wherein the plurality of proteins includes at least one of a DNA repair protein, a cell cycle protein, and a protein encoded by a cancer driver gene.

4. The method of any one of the preceding claims, wherein the transcriptomic data is RNAseq data.

5. The method of any one of the preceding claims, wherein the step of stratifying uses a cutoff value that is optimized for a ratio between true positive and false negative.

6. The method of any one of the preceding claims, wherein the derived phenotype of the cancer tissue is TNBC.

7. The method of any one of the preceding claims, wherein the step of clustering uses between 3 and 10 clusters.

8. The method of any one of the preceding claims, wherein the recursive feature elimination is repeated at least once.

9. The method of any one of the preceding claims, wherein the reduced transcriptomic data are less than 30% of the transcriptomic data of the cancer tissue.

10. The method of any one of the preceding claims, wherein the reduced transcriptomic data is less than 10% of the transcriptomic data of the cancer tissue.

11. The method of any one of the preceding claims, wherein the reduced transcriptomic data is less than 1% of the transcriptomic data of the cancer tissue.

12. The method of any one of the preceding claims, further comprising a step of associating the reduced transcriptomic data to at least one of a drug response, overall survival, disease free survival, and progression free survival.

13. The method of any one of the preceding claims, further comprising a step of using the reduced transcriptomic data as input for a pathway analysis.

14. The method of claim 12, further comprising a step of determining a treatment regimen based on at least one of the drug response, the overall survival, the disease free survival, and the progression free survival.

15. The method of claim 14, further comprising treating a patient having the cancer tissue with a cancer treatment in the treatment regimen in a dose and a schedule sufficient to treat the cancer tissue.

16. The method of claim 1, wherein the transcriptomic data is RNAseq data.

17. The method of claim 1, wherein the step of stratifying uses a cutoff value that is optimized for a ratio between true positive and false negative.

18. The method of claim 1, wherein the derived phenotype of the cancer tissue is TNBC.

19. The method of claim 1, wherein the step of clustering uses between 3 and 10 clusters.

20. The method of claim 1, wherein the recursive feature elimination is repeated at least once.

21. The method of claim 1, wherein the reduced transcriptomic data are less than 30% of the transcriptomic data of the cancer tissue.

22. The method of claim 1, wherein the reduced transcriptomic data is less than 10% of the transcriptomic data of the cancer tissue.

23. The method of claim 1, wherein the reduced transcriptomic data is less than 1% of the transcriptomic data of the cancer tissue.

24. The method of claim 1, further comprising a step of associating the reduced transcriptomic data to at least one of a drug response, overall survival, disease free survival, and progression free survival.

25. The method of claim 1, further comprising a step of using the reduced transcriptomic data as input for a pathway analysis.

26. The method of claim 24, further comprising a step of determining a treatment regimen based on at least one of the drug response, the overall survival, the disease free survival, and the progression free survival.

27. The method of claim 26, further comprising treating a patient having the cancer tissue with a cancer treatment in the treatment regimen in a dose and a schedule sufficient to treat the cancer tissue.

28. A system for processing omics data of a cancer tissue, comprising:
an omics database storing transcriptomic data of the cancer tissue; and
a machine learning system informationally coupled to the omics database and programmed to:
obtain the transcriptomic data of the cancer tissue, wherein the transcriptomics data is associated with protein expression level of a plurality of proteins in the cancer tissue, and wherein the plurality of proteins is associated with a phenotype of the cancer tissue;
stratify the transcriptomics data into a subgroup of data, and clustering the subgroup of data; and
subject the clustered subgroup of data to recursive feature elimination to obtain reduced transcriptomic data.

29. The system of claim 28, wherein the cancer sample is a breast cancer sample, and in which the plurality of proteins includes at least one of an estrogen receptor, a progesterone receptor, and HER2.

30. The system of claim 28, wherein the plurality of proteins includes at least one of a DNA repair protein, a cell cycle protein, and a protein encoded by a cancer driver gene.

31. The system of any one of claims 28-30, wherein the transcriptomic data is RNAseq data.

32. The system of any one of claims 28-31, wherein the transcriptomics data is stratified using a cutoff value that is optimized for a ratio between true positive and false negative.

33. The system of any one of claims 28-32, wherein the derived phenotype of the cancer tissue is TNBC.

34. The system of any one of claims 28-33, wherein the subgroup is clustered using between 3 and 10 clusters.

35. The system of any one of claims 28-34, wherein the recursive feature elimination is repeated at least once.

36. The system of any one of claims 28-35, wherein the reduced transcriptomic data are less than 30% of the transcriptomic data of the cancer tissue.

37. The system of any one of claims 28-36, wherein the reduced transcriptomic data is less than 10% of the transcriptomic data of the cancer tissue.

38. The system of any one of claims 28-37, wherein the reduced transcriptomic data is less than 1% of the transcriptomic data of the cancer tissue.

39. The system of any one of claims 28-38, wherein the machine learning system is further programmed to associate the reduced transcriptomic data to at least one of a drug response, overall survival, disease free survival, and progression free survival.

40. The system of any one of claims 28-39, wherein the machine learning system is further programmed to use the reduced transcriptomic data as input for a pathway analysis.

41. The system of claim 40, wherein the machine learning system is further programmed to determine a treatment regimen based on at least one of the drug response, the overall survival, the disease free survival, and the progression free survival.

42. A non-transient computer readable medium containing program instructions for causing a computer system comprising a machine learning system to perform a method, wherein the machine learning system is informationally coupled to an omics database that stores transcriptomic data of a cancer tissue, wherein the method comprises the steps of:
obtaining the transcriptomic data of the cancer tissue, wherein the transcriptomics data is associated with protein expression level of a plurality of proteins in the cancer tissue, and wherein the plurality of proteins is associated with a phenotype of the cancer tissue;
stratifying the transcriptomics data into a subgroup of data, and clustering the subgroup of data; and
subjecting the clustered subgroup of data to recursive feature elimination to obtain reduced transcriptomic data.

43. The non-transient computer readable medium of claim 42, wherein the cancer sample is a breast cancer sample, and in which the plurality of proteins includes at least one of an estrogen receptor, a progesterone receptor, and HER2.

44. The non-transient computer readable medium of claim 42, wherein the plurality of proteins includes at least one of a DNA repair protein, a cell cycle protein, and a protein encoded by a cancer driver gene.

45. The non-transient computer readable medium of any of claims 42-44, wherein the transcriptomic data is RNAseq data.

46. The non-transient computer readable medium of any of claims 42-45, wherein the step of stratifying uses a cutoff value that is optimized for a ratio between true positive and false negative.

47. The non-transient computer readable medium of any of claims 42-46, wherein the derived phenotype of the cancer tissue is TNBC.

48. The non-transient computer readable medium of any of claims 42-47, wherein the step of clustering uses between 3 and 10 clusters.

49. The non-transient computer readable medium of any of claims 42-48, wherein the recursive feature elimination is repeated at least once.

50. The non-transient computer readable medium of any of claims 42-49, wherein the reduced transcriptomic data are less than 30% of the transcriptomic data of the cancer tissue.

51. The non-transient computer readable medium of any of claims 42-50, wherein the reduced transcriptomic data is less than 10% of the transcriptomic data of the cancer tissue.

52. The non-transient computer readable medium of any of claims 42-51, wherein the reduced transcriptomic data is less than 1% of the transcriptomic data of the cancer tissue.

53. The non-transient computer readable medium of any of claims 42-52, wherein the method further comprises a step of associating the reduced transcriptomic data to at least one of a drug response, overall survival, disease free survival, and progression free survival.

54. The non-transient computer readable medium of any of claims 42-53, further comprising a step of using the reduced transcriptomic data as input for a pathway analysis.

55. The non-transient computer readable medium of claim 53, wherein the method further comprises a step of determining a treatment regimen based on at least one of the drug response, the overall survival, the disease free survival, and the progression free survival.
